(19) United States
(12) Patent Application Publication
(10) Pub. No.: US 2010/0021396 A1
(43) Pub. Date: Jan. 28, 2010

(54) TINTING MOUSSE

(75) Inventors: Astrid Kleen, Hamburg (DE); Stephan Schwartz, Wedel (DE); Susanne Bietz, Elmshorn (DE); Maureen Rippe, Schneeverdingen (DE); Sabrina Zirwen, Hamburg (DE)

Correspondence Address: CONNOLLY BOVE LODGE & HUTZ LLP, PO BOX 2207, WILMINGTON, DE, 19899-2207, US

(73) Assignee: Henkel AG & Co. KGaA, Duesseldorf (DE)

(21) Appl. No.: 12/444,858

(22) PCT Filed: Aug. 8, 2007
(86) PCT No.: PCT/EP2007/058216

§ 371 Date: Apr. 9, 2009

(30) Foreign Application Priority Data
Oct. 12, 2006 (DE) 10 2006 048 740.0

(51) Int. Cl. A61K 8/72; A61Q 5/06

(52) U.S. Cl. 424/47

(57) ABSTRACT

Aerosol-foam products or pump-foam products for simultaneous coloring or tinting and temporary deformation of keratinic fibers, encompassing a foam-type or foamable composition that contains at least one film-forming and/or setting polymer and at least one anionic direct dye.

The present invention relates to aerosol-foam products or pump-foam products for simultaneous coloring or tinting and temporary deformation of keratinic fibers, encompassing a foam-type or foamable composition that contains at least one film-forming and/or setting polymer and at least one anionic direct dye.

“Keratin-containing” fibers are understood in principle as all animal hairs, e.g. wool, horsehair, angora wool, furs, feathers, and products or textiles produced therefrom. By preference, however, the keratinic fibers are human hairs.

An attractive-looking hairstyle is generally regarded these days as an indispensable element of a well-groomed appearance. Present-day fashion trends are such that more and more hairstyles regarded as chic are ones that, for many types of hair, can be constructed, or maintained for a longer period of time of up to several days, only with the use of setting ingredients. Hair treatment agents that serve for permanent or temporary shaping of the hair therefore play an important role. Temporary shaping actions that are intended to yield good hold without impairing the hair's healthy appearance, for example its shine, can be achieved, for example, using hair sprays, hair waxes, hair gels, setting foams, blow-dry waves, etc.

The desire to create a certain hairstyle is often also accompanied by the desire to permanently or temporarily modify one's natural hair color. A large number of special products are offered for achieving both goals with only one hair treatment; these are referred to, for example, as coloring setting agents or tinting setting agents.

Corresponding agents usually contain synthetic polymers as a shaping component, and so-called direct dyes as color-modifying substances. These are dye molecules that absorb directly onto the hair and do not require an oxidizing process in order to form the color. Included among these dyes are, for example, henna, which has been known since antiquity for coloring the hair and body.

DE 196 51 482 C1 discloses setting agents for coloring and tinting human hair that contain at least one nonionic polymer, if applicable mixed with a cationic polymer, and at least one anionic surfactant. The agents further contain direct dyes, all usual physiologically unobjectionable direct dyes being used. According to claim 4, cationic dyes are used by preference.

DE 201 00 857 U1 describes gel-type hair coloring agents that contain at least one direct dye, at least one long-chain quaternary ammonium compound, at least one cationic polymer, at least one nonionic and/or amphoteric or zwitterionic polymer, 15 to 50 wt % of at least one lower alcohol, and water, and exhibit a certain viscosity. All known cationic dyes can be used as direct dyes. The use of direct vegetable dyes or anionic dyes is also recited as possible.

It has now been found that, especially with regard to the formulation of tinting setting agents as foam-type or foamable agents, problems often occur in terms of the stability of the dyes used in the particular cosmetic carrier, as well as problems with the stability of the agent.

The object of the present invention was therefore to make available aerosol-foam products or pump-foam products for simultaneous coloring or tinting and temporary deformation of keratinic fibers, in which products the aforesaid stability problems do not arise. A particular intention was to develop stable products that contain at least one direct dye of the blue and/or violet color tendency and that permit maximally neutral color results. The products were also intended to permit the gentlest possible treatment of the keratinic fibers.

It has now been found that this can be achieved by the use of special direct dyes.

The subject matter of the present invention is therefore an aerosol-foam product or pump-foam product for the treatment of keratinic fibers, encompassing a foam-type or foamable composition containing, in a cosmetically acceptable carrier,
a) at least one film-forming and/or setting polymer, and
b) at least one anionic direct dye.

Surprisingly, anionic direct dyes in cosmetically acceptable carriers, such as those usually used for the formulation of foam-type or foamable compositions, are notable for particularly high stability. Even in the presence of cationic ingredients, both the dyes and the composition remain inherently stable.

The products according to the present invention contain at least one film-forming and/or setting polymer. The product according to the present invention contains the film-forming and/or setting polymer as a rule in a quantity from 0.1 to 20 wt %, based on the entire foam-type or foamable composition, by preference in a quantity from 0.5 to 15 wt %, particularly preferably from 1 to 10 wt %. Several film-forming and/or setting polymers can of course also be contained. The total quantity of film-forming and/or setting polymers is by preference at most 30 wt %, based on the entire foam-type or foamable composition.

These film-Forming and/or setting polymers can be both permanently and temporarily cationic, anionic, nonionic, or amphoteric. When at least two film-forming and/or setting polymers are used, they can of course have different charges. It may be preferred according to the present invention if an ionic film-forming and/or setting polymer is used together with an amphoteric and/or nonionic film-forming and/or setting polymer. The use of at least two oppositely charged film-forming and/or setting polymers is also preferred. In the latter case, a particular embodiment can in turn additionally contain at least one further amphoteric and/or nonionic film-forming and/or setting polymer.

Because polymers are often multifunctional, their functions cannot always be clearly and unequivocally distinguished from one another. This applies in particular to film-forming and setting polymers. It is explicitly stated at this juncture, however, that in the context of the present invention, both film-forming and setting polymers are essential. Because the two properties are also not entirely independent of one another, the term “setting polymers” is also always understood as “film-forming polymers,” and vice versa.

Included among the preferred properties of the film-forming polymers is film formation. “Film-forming polymers” are to be understood as those polymers that, upon drying, leave behind a continuous film on the skin, hair, or nails. Film-formers of this kind can be used in a very wide variety of cosmetic products such as, for example, face masks, make-up, hair setting agents, hair sprays, hair gels, hair waxes, hair therapies, shampoos, or nail polishes. Particularly preferred are those polymers that possess sufficient solubility in alcohol or in water/alcohol mixtures to be present in completely dissolved form in the agent according to the present invention. The film-forming polymers can be of synthetic or natural origin.

“Film-forming polymers” are furthermore understood according to the present invention to be those polymers that, when used in a 0.01 to 20-wt % aqueous, alcoholic, or aqueous/alcoholic solution, are capable of depositing a transparent polymer film on the hair. The film-forming polymers can be anionically, amphoterically, nonionically, permanently cationically, or temporarily cationically charged.

Suitable synthetic film-forming, hair-setting polymers are homo- or copolymers that are constructed from at least one of the following monomers: vinylpyrrolidone, vinyl caprolactam, vinyl esters such as, for example, vinyl acetate, vinyl alcohol, acrylamide, methacrylamide, alkyl and dialkyl acrylamide, alkyl and dialkyl methacrylamide, alkyl acrylate, alkyl methacrylate, propylene glycol or ethylene glycol, the alkyl groups of these monomers being by preference C₁ to C₇ alkyl groups, particularly preferably C₁ to C₃ alkyl groups.

Mention may be made, by way of example, of homopolymers of vinyl caprolactam, vinylpyrrolidone or N-vinylformamide. Further suitable synthetic film-forming, hair-setting polymers are, for example, copolymers of vinylpyrrolidone and vinyl acetate, terpolymers of vinylpyrrolidone, vinyl acetate, and vinyl propionate, polyacrylamides that are marketed, for example, under the commercial names Akypomine® P 191 of the CHEM-Y company, Emmerich, or Sepigel® 305 of the Seppic company; polyvinyl alcohols that are marketed, for example, under the commercial names Elvanol® of DuPont or Vinol® 523/540 of the Air Products company, and polyethylene glycol/polypropylene glycol copolymers that are marketed, for example, under the commercial designations Ucon® of Union Carbide.

Suitable natural film-forming polymers are, for example, cellulose derivatives, for example hydroxypropyl cellulose having a molecular weight from 30,000 to 50,000 g/mol, which is marketed for example under the commercial name Nisso SI® by the Lehmann & Voss company, Hamburg.

Setting polymers contribute to the hold, and/or to building up the hair volume and hair fullness, of the overall hairstyle. These so-called setting polymers are at the same time also film-forming polymers, and are therefore generally typical substances for shaping hair-treatment agents such as hair setting agents, hair foams, hair waxes, hair sprays. It is certainly possible for film formation to be localized, and for only a few fibers to be connected to one another.

Substances that furthermore impart hydrophobic properties to the hair are preferred in this context, since they decrease the hair's tendency to absorb humidity, i.e. water. This decreases loose hanging of strands of hair, and thus ensures long-term hairstyle construction and retention. The so-called “curl retention” test is often used as a test method for this. These polymeric substances can furthermore be successfully incorporated into leave-on and rinse-off hair therapies or shampoos. Because polymers are often multifunctional, i.e. exhibit multiple effects that are desirable in terms of applications engineering, numerous polymers fall into multiple groups categorized in terms of effect; this is also the case in the CFTA handbook. Because of the importance of the setting polymers in particular, these will be listed explicitly in the form of their INCI names. The aforesaid film-forming polymers are thus also, of course, specifically featured in this list of the polymers to be used in preferred fashion according to the present invention.

Examples of commonly used film-forming setting polymers are Acrylamide/Ammonium Acrylate Copolymer, Acrylamides/DMAPA Acrylates/Methoxy PEG Methacrylate Copolymer, Acrylamidopropyltrimonium Chloride/Acrylamide Copolymer, Acrylamidopropyltrimonium Chloride/Acrylates Copolymer, Acrylates/Acetoacetoxyethyl Methacrylate Copolymer, Acrylates/Acrylamide Copolymer, Acrylates/Ammonium Methacrylate Copolymer, Acrylates/t-Butylacrylamide Copolymer, Acrylates Copolymer, Acrylates/C1-2 Succinates/Hydroxyacrylates Copolymer, Acrylates/Lauryl Acrylate/Stearyl Acrylate/Ethylamine Oxide Methacrylate Copolymer, Acrylates/Octylacrylamide Copolymer, Acrylates/Octylacrylamide/Diphenyl Amodimethicone Copolymer, Acrylates/Stearyl Acrylate/Ethylamine Oxide Methacrylate Copolymer, Acrylates/VA Copolymer, Acrylates/VP Copolymer, Adipic Acid/Diethylenetriamine Copolymer, Adipic Acid/Dimethylaminohydroxypropyl Diethylenetriamine Copolymer, Adipic Acid/Epoxypropyl Diethylenetriamine Copolymer, Adipic Acid/Isophthalic Acid/Neopentyl Glycol/Trimethylolpropane Copolymer, Allyl Stearate/VA Copolymer, Aminoethylacrylate Phosphate/Acrylates Copolymer, Aminoethylpropanediol-Acrylates/Acrylamide Copolymer, Aminoethylpropanediol-AMPD-Acrylates/Diacetoneacrylamide Copolymer, Ammonium VA/Acrylates Copolymer, AMPD-Acrylates/Diacetoneacrylamide Copolymer, AMP-Acrylates/Allyl Methacrylate Copolymer, AMP-Acrylates/C1-18 Alkyl Acrylates/C1-8 Alkyl Acrylamide Copolymer, AMP-Acrylates/Diacetoneacrylamide Copolymer, AMP-Acrylates/Dimethylaminoethylmethacrylate Copolymer, Bacillus/Rice Bran Extract/Soybean Extract Ferment Filtrate, Bis-Butyloxyamodimethicone/PEG-60 Copolymer, Butyl Acrylate/Ethylhexyl Methacrylate Copolymer, Butyl Acrylate/Hydroxypropyl Dimethicone Acrylate Copolymer, Butylated PVP, Butyl Ester of Ethylene/MA Copolymer, Butyl Ester of PVM/MA Copolymer, Calcium/Sodium PVM/MA Copolymer, Corn Starch/Acrylamide/Sodium Acrylate Copolymer, Diethylene Glycolamine/Epichlorohydrin/Piperazine Copolymer, Dimethicone Crosspolymer, Diphenyl Amodimethicone, Ethyl Ester of PVM/MA Copolymer, Hydrolyzed Wheat Protein/PVP Crosspolymer, Isobutylene/Ethylmaleimide/Hydroxyethylmaleimide Copolymer, Isobutylene/MA Copolymer, Isobutylmethacrylate/Bis-Hydroxypropyl Dimethicone Acrylate Copolymer, Isopropyl Ester of PVM/MA Copolymer, Lauryl Acrylate Crosspolymer, Lauryl Methacrylate/Glycol Dimethacrylate Crosspolymer, MEA-Sulfite, Methacrylic Acid/Sodium Acrylamidomethyl Propane Sulfonate Copolymer, Methacryloyl Ethyl Betaine/Acrylates Copolymer, Octylacrylamide/Acrylates/Butylaminoethyl Methacrylate Copolymer, PEG/PPG-25/25 Dimethicone/Acrylates Copolymer, PEG-8/SMDI Copolymer, Polyacrylamide, Polyacrylate-6, Polybeta-Alanine/Glutaric Acid Crosspolymer, Polybutylene Terephthalate, Polyester-1, Polyethylacrylate, Polyethylene Terephthalate, Polymethacryloyl Ethyl Betaine, Polypentaerythrityl Terephthalate, Polyperfluoroperhydrophenanthrene, Polyquaternium-1, Polyquaternium-2, Polyquaternium-4, Polyquaternium-5, Polyquaternium-6, Polyquaternium-7, Polyquaternium-8, Polyquaternium-9, Polyquaternium-10, Polyquaternium-11, Polyquaternium-12, Polyquaternium-13, Polyquaternium-14, Polyquaternium-15, Polyquaternium-16, Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-30, Polyquaternium-31, Polyquaternium-32, Polyquaternium-33, Polyquaternium-34, Polyquaternium-35, Polyquaternium-36, Polyquaternium-37, Polyquaternium-39, Polyquaternium-45, Polyquaternium-46, Polyquaternium-47, Polyquaternium-48, Polyquaternium-49, Polyquaternium-50, Polyquaternium-55, Polyquaternium-56, Polysilicone-9, Polyurethane-1, Polyurethane-6, Polyurethane-10, Polyvinyl Acetate, Polyvinyl Butyral, Polyvinylcaprolactam, Polyvinylformamide, Polyvinyl Imidazolinium Acetate, Polyvinyl Methyl Ether, Potassium Butyl Ester of PVM/MA Copolymer, Potassium Ethyl Ester of PVM/MA Copolymer, PPG-70 Polyglyceryl-10 Ether, PPG-12/SMDI Copolymer, PPG-51/SMDI Copolymer, PPG-10 Sorbitol, PVM/MA Copolymer, PVP, PVP/VA/Itaconic Acid Copolymer, PVP/VA/Vinyl Propionate Copolymer, Rhizobian Gum, Rosin Acrylate, Shellac, Sodium Butyl Ester of PVM/MA Copolymer, Sodium Ethyl Ester of PVM/MA Copolymer, Sodium Polyacrylate, Sterculia Urens Gum, Terephthalic Acid/Isophthalic Acid/Sodium Isophthalic Acid Sulfonate/Glycol Copolymer, Trimethylolpropane Triacrylate, Trimethylsiloxysilylcarbamoyl Pullulan, VA/Crotonates Copolymer, VA/Crotonates/Methacryloxybenzophenone-1 Copolymer, VA/Crotonates/Vinyl Neodecanoate Copolymer, VA/Crotonates/Vinyl Propionate Copolymer, VA/DBM Copolymer, VA/Vinyl Butyl Benzoate/Crotonates Copolymer, Vinylamine/Vinyl Alcohol Copolymer, Vinyl Caprolactam/VP/Dimethylaminoethyl Methacrylate Copolymer, VP/Acrylates/Lauryl Methacrylate Copolymer, VP/Dimethylaminoethylmethacrylate Copolymer, VP/DMAPA Acrylates Copolymer, VP/Hexadecene Copolymer, VP/VA Copolymer, VP/Vinyl Caprolactam/DMAPA Acrylates Copolymer, Yeast Palmitate.

Products according to the present invention whose foam-type or foamable composition contains at least one nonionic film-forming and/or setting polymer are preferred.

The nonionic film-forming and/or setting polymer is by preference a homo- or copolymer of vinylpyrrolidone, particularly preferably polyvinylpyrrolidone and/or a vinylpyrrolidone/vinyl acetate copolymer.

In a further preferred embodiment, the foam-type or foamable composition contains at least one cationic and/or setting polymer.

The cationic film-forming and/or setting polymer is by preference a quaternized homo- or copolymer of dimethyldiallylammonium chloride or a copolymer of vinylpyrrolidone having at least one further monomer, which if applicable contains at least one cationic group after quaternization.

Particularly preferably, the foam-type or foamable compositions contain at least one nonionic and at least one cationic film-forming and/or setting polymer.

The products according to the present invention furthermore contain at least one direct dye. Direct dyes are usually nitrophenylendiamines, nitroaminophenols, azo dyes, anthraquinones, or indophenols. The direct dyes are used preferably in a quantity from 0.001 to 20 wt %, based on the foam-type or foamable compositions, i.e. a propellant component that may be present in the product according to the present invention remained unaccounted for in terms of the quantitative indication. The total quantity of direct dyes is by preference at most 20 wt %, based on the foam-type or foamable compositions.

According to the present invention, at least one anionic direct dye is used.

All anionic direct dyes that are known and approved for hair coloring agents are suitable in principle, in particular 6-hydroxy-5-[(4-sulfophenyl)azo]-2-naphthalenesulfonic acid disodium salt (C.I. 15,985; Food Yellow No. 3; FD&C Yellow No. 6), 2,4-dinitro-1-naphthol-7-sulfonic acid disodium salt (C.I. 10,316; Acid Yellow 1; Food Yellow No. 1), 2-(indane-1,3-dion-2-yl)quinoline-x,x-sulfonic acid (mixture of mono- and disulfonic acid) (C.I. 47,005; D&C Yellow No. 10; Food Yellow No. 13; Acid Yellow 3, Yellow 10), 4-((4-amino-3-sulfophenyl)azo)benzenesulfonic acid disodium salt (C.I. 13,015, Acid Yellow 9), 5-hydroxy-1-(4-sulfophenyl)-4-[(4-sulfophenyl)azo]pyrazole-3-carboxylic acid trisodium salt (C.I. 19,140; Food Yellow No. 4; Acid Yellow 23), 3-[(4-phenylamino)phenyl]azobenzenesulfonic acid sodium salt (C.I. 13,065; Ki406; Acid Yellow 36), 9-(2-carboxyphenyl)-6-hydroxy-3H-xanthen-3-one (C.I. 45,350; Acid Yellow 73; D&C Yellow No. 8), 5-[(2,4-dinitrophenyl)amino]-2-phenylaminobenzenesulfonic acid sodium salt (C.I. 10,385; Acid Orange 3), 4-[(2,4-dihydroxyphenyl)azo]-benzenesulfonic acid sodium salt (C.I. 14,270; Acid Orange 6), 4-[(2-hydroxynaphth-1-yl)azo]-benzenesulfonic acid sodium salt (C.I. 15,510; Acid Orange 7), 4-[(2,4-dihydroxy-3-[(2,4-dimethylphenyl)azo]-phenyl)azo]-benzenesulfonic acid sodium salt (C.I. 20,1701 Acid Orange 24), 4-hydroxy-3-[(2-methoxyphenyl)azo]-1-naphthalenesulfonic acid sodium salt (C.I. 14,710; Acid Red 4), 4-hydroxy-3-[(4-sulfonaphth-1-yl)azo]-1-naphthalenesulfonic acid disodium salt (C.I. 14,720; Acid Red No. 14), 6-hydroxy-5-[(4-sulfonaphth-1-yl)azo]-2,4-naphthalenedisulfonic acid trisodium salt (C.I. 16,255; Ponceau 4R; Acid Red 18), 3-hydroxy-4-[(4-sulfonaphth-1-yl)azo]-2,7-naphthalenedisulfonic acid trisodium salt (C.I. 16,185; Acid Red 27), 8-amino-1-hydroxy-2-(phenylazo)-3,6-naphthalenedisulfonic acid disodium salt (C.I. 17,200; Acid Red 33; Red 33), 5-(acetylamino)-4-hydroxy-3-[(2-methylphenyl)azo]-2,7-naphthalenedisulfonic acid disodium salt (C.I. 18,065; Acid Red 35), 2-(3-hydroxy-2,4,5,7-tetraiododibenzopyran-6-on-9-yl)-benzoic acid disodium salt (C.I. 45,430; Acid Red 51), N-[6-(diethylamino)-9-(2,4-disulfophenyl)-3H-xanthen-3-ylidene]-N-ethylethanammonium hydroxide, internal salt, sodium salt (C.I. 45,100; Acid Red 52), 8-[(4-(phenylazo)phenyl)azo]-7-naphthol-1,3-disulfonic acid disodium salt (C.I. 27,290; Acid Red 73), 2′,4′,5′,7′-tetrabromo-3′,6′-dihydroxyspiro[isobenzofuran-1(3H),9′-[9H]xanthen]-3-one disodium salt (C.I. 45,380; Acid Red 87), 2′,4′,5′,7′-tetrabromo-4,5,6,7-tetrachloro-3′,6′-dihydroxyspiro[isobenzofuran-1(3H),9′[9H]xanthen]-3-one disodium salt (C.I. 45,410; Acid Red 92), 3′,6′-dihydroxy-4′,5′-diiodospiro[isobenzofuran-1(3H), 9′(9H)-xanthen]-3-one disodium salt (C.I. 45425; Acid Red 95), 2-hydroxy-3-((2-hydroxynaphth-1-yl)azo)-5-nitrobenzenesulfonic acid sodium salt (C.I. 15,685; Acid Red 184), 3-hydroxy-4-(3-methyl-5-oxo-1-phenyl-4,5-dihydro-1H-pyrazol-4-ylazo)-naphthalene-1-sulfonic acid sodium salt, chromium complex (Acid Red 195), 3-hydroxy-4-[(4-methyl-2-sulfophenyl)azo]-2-naphthalenecarboxylic acid calcium salt (C.I. 15,850:1; Pigment Red 57:1), 3-[(2,4-dimethyl-5-sulfophenyl)azo]-4-hydroxy-1-naphthalenesulfonic acid disodium salt (C.I. 14,700; Food Red No. 1; Ponceau SX; FD&C Red No. 4), 1,4-bis[(2-sulfo-4-methylphenyl)amino]-9,10-anthraquinone disodium salt (C.I. 61,570; Acid Green 25), bis[4-(dimethylamino)phenyl]-(3,7-disulfo-2-hydroxynaphth-1-yl)carbenium internal salt, sodium salt (C.I. 44,090; Food Green No. 4; Acid Green 50), bis[4-(diethylamino)-phenyl](2,4-disulfophenyl)carbenium internal salt, sodium salt (2:1) (C.I. 42,045; Food Blue No. 3; Acid Blue 1), bis[4-(diethylamino)phenyl](5-hydroxy-2,4-disulfophenyl)carbenium internal salt, calcium salt (2:1) (C.I. 42,051; Acid Blue 3), N-[4-[(2,4-disulfophenyl)[4-[ethyl(phenylmethyl)amino)phenyl]methylene]-2,5-cyclohexadiene-1-ylidene]-N-ethylbenzenemethanaminium hydroxide, internal salt, sodium salt (C.I. 42,080; Acid Blue 7), (2-sulfophenyl)di[4-(ethyl((4-sulfophenyl)methyl)amino)phenyl]-carbenium disodium salt betaine (C.I. 42,090; Acid Blue 9; FD&C Blue No. 1), 1-amino-4-(phenylamino)-9,10-anthraquinone-2-sulfonic acid (C.I. 62,055; Acid Blue 25), 1-amino-4-(cyclohexylamino)-9,10-anthraquinone-2-sulfonic acid sodium salt (C.I 62,045; Acid Blue 62), 2-(1,3-dihydro-3-oxo-5-sulfo-2H-indol-2-ylidene)-2,3-dihydro-3-oxo-1H-indole-5-sulfonic acid disodium salt (C.I. 73,015; Acid Blue 74), 9-(2-carboxyphenyl)-3-[(2-methylphenyl)amino]-6-[(2-methyl-4-sulfophenyl)amino]xanthylium internal salt, sodium salt (C.I. 45,190; Acid Violet 9), 1-hydroxy-4-[(4-methyl-2-sulfophenyl)amino]-9,10-anthraquinone sodium salt (C.I. 60,730; D&C Violet No. 2; Acid Violet 43), bis[3-nitro-4-[(4-phenylamino)-3-sulfophenylamino]phenyl]sulfone (C.I. 10,410; Acid Brown 13), 5-amino-4-hydroxy-6-[(4-nitrophenyl)azo]-3-(phenylazo)-2,7-naphthalenedisulfonic acid disodium salt (C.I. 20,470; Acid Black 1), 3-hydroxy-4-[(2-hydroxynaphth-1-yl)azo]-7-nitro-1-naphthalenesulfonic acid chromium complex (3:2) (C.I. 15,711; Acid Black 52), 4-(acetylamino)-5-hydroxy-6-[(7-sulfo-4-[(4-sulfophenyl)azo]naphth-1-yl)azo]-1,7-naphthalenedisulfonic acid tetrasodium salt (C.I. 28,440; Food Black No. 1), 3′,3″,5′,5″-tetrabromophenolsulfonphthalein (bromophenol blue).

The products according to the present invention by preference contain at least one anionic direct dye selected from blue- or violet-coloring dyes, particularly preferably selected from bis[4-(diethylamino)-phenyl](2,4-disulfophenyl)carbenium internal salt, sodium salt (2:1) (C.I. 42,045; Food Blue No. 3; Acid Blue 1), bis[4-(diethylamino)phenyl](5-hydroxy-2,4-disulfophenyl)carbenium internal salt, calcium salt (2:1) (C.I. 42,051; Acid Blue 3), N-[4-[(2,4-disulfophenyl)[4-[ethyl(phenylmethyl)amino)phenyl]methylene]-2,5-cyclohexadien-1-ylidene]-N-ethylbenzenemethanaminium hydroxide, internal salt, sodium salt (C.I. 42,080; Acid Blue 7), (2-sulfophenyl)di[4-(ethyl((4-sulfophenyl)methyl)amino)phenyl]carbenium disodium salt betaine (C.I. 42,090; Acid Blue 9; FD&C Blue No. 1), 1-amino-4-(phenylamino)-9,10-anthraquinone-2-sulfonic acid (C.I. 62,055; Acid Blue 25), 1-amino-4-(cyclohexylamino)-9,10-anthraquinone-2-sulfonic acid sodium salt (C.I. 62,045; Acid Blue 62), 2-(1,3-dihydro-3-oxo-5-sulfo-2H-indol-2-ylidene)-2,3-dihydro-3-oxo-1H-indole-5-sulfonic acid disodium salt (C.I. 73,015; Acid Blue 74), 9-(2-carboxyphenyl)-3-[(2-methylphenyl)amino]-6-[(2-methyl-4-sulfophenyl)amino]xanthylium internal salt, sodium salt (C.I. 45,190; Acid Violet 9), 1-hydroxy-4-[(4-methyl-2-sulfophenyl)amino]-9,10-anthraquinone sodium salt (C.I. 60,730; D&C Violet No. 2; Acid Violet 43), 3′,3″,5′,5″-tetrabromophenolsulfonphthalein (bromophenol blue), and 5-amino-4-hydroxy-6-[(4-nitrophenyl)azo]-3-(phenylazo)-2,7-naphthalenedisulfonic acid disodium salt (C.I. 20,470; Acid Black 1).

Particularly preferably, the products according to the present invention contain at least one anionic direct dye selected from (2-sulfophenyl)di[4-(ethyl((4-sulfophenyl)methyl)amino)phenyl]carbenium disodium salt betaine (C.I. 42,090; Acid Blue 9; FD&C Blue No. 1), 1-amino4-(cyclohexylamino)-9,10-anthraquinone-2-sulfonic acid sodium salt (C.I. 62,045; Acid Blue 62), 1-hydroxy-4-[(4-methyl-2-sulfophenyl)amino]-9,10-anthraquinone sodium salt (C.I. 60,730; D&C Violet No. 2; Acid Violet 43), and 5-amino-4-hydroxy-6-[(4-nitrophenyl)azo]-3-(phenylazo)-2,7-naphthalenedisulfonic acid disodium salt (C.I. 20,470; Acid Black 1).

Very particularly preferably, the products according to the present invention contain the anionic direct dye 1-hydroxy-4-[(4-methyl-2-sulfophenyl)amino]-9,10-anthraquinone sodium salt (C.I. 60,730; D&C Violet No. 2; Acid Violet 43).

The use of the aforesaid blue- or violet-coloring dyes permits color results that are notable, in particular, for a neutral color tendency.

By preference, at least one cationic direct dye is also used alongside at least one anionic direct dye; once again, all dyes known and approved for hair coloring agents are suitable in principle.

Suitable, for example, are 9-(dimethylamino)benzo[a]phenoxazin-7-ium chloride (C.I. 51,175; Basic Blue 6), di[4-(diethylamino)phenyl][4-(ethylamino)naphthyl]carbenium chloride (C.I. 42,595; Basic Blue 7), di-(4-(dimethylamino)phenyl)-(4-(methylphenylamino)naphthalen-1-yl)carbenium chloride (C.I. 42,563; Basic Blue 8), 3,7-di(dimethylamino)phenothiazin-5-ium chloride (C.I. 52,015 Basic Blue 9), di[4-(dimethylamino)phenyl][4-(phenylamino)naphthyl]carbenium chloride (C.I. 44,045; Basic Blue 26), 2-[(4-(ethyl(2-hydroxyethyl)amino)phenyl)azo]-6-methoxy-3-methylbenzothiazolium methylsulfate (C.I. 11,154; Basic Blue 41), 8-amino-2-bromo-5-hydroxy-4-imino-6-[(3-(trimethylammonio)phenyl)amino]-1(4H)-naphthalenone chloride (C.I. 56,059; Basic Blue No. 99), bis[4-(dimethylamino)phenyl]-[4-(methylamino)phenyl]carbenium chloride (C.I. 42,535; Basic Violet 1), tri(4-amino-3-methylphenyl)carbenium chloride (C.I. 42,520; Basic Violet 2), tri[4-(dimethylamino)phenylcarbenium chloride (C.I. 42,555; Basic Violet 3), 2-[3,6-(diethylamino)dibenzopyranium-9-yl]-benzoic acid chloride (C.I. 45,170; Basic Violet 10), di(4-aminophenyl)(4-amino-3-methylphenyl)carbenium chloride (C.I. 42,510 Basic Violet 14), 1,3-bis[(2,4-diamino-5-methylphenyl)azo]-3-methylbenzene (C.I. 21,010; Basic Brown 4), 1-[(4-aminophenyl)azo]-7-(trimethylammonio)-2-naphthol chloride (C.I. 12,250; Basic Brown 16), 1-[(4-amino-2-nitrophenyl)azo]-7-(trimethylammonio)-2-naphthol chloride, 1-[(4-amino-3-nitrophenyl)azo]-7-(trimethylammonio)-2-naphthol chloride (C.I. 12,251; Basic Brown 17), 3-[(4-amino-2,5-dimethoxyphenyl)azo]-N,N,N-trimethylbenzeneaminium chloride (C.I. 12,605, Basic Orange 69), 3,7-diamino-2,8-dimethyl-5-phenylphenazinium chloride (C.I. 50,240; Basic Red 2), 1,4-dimethyl-5-[(4-(dimethylamino)phenyl)azo]-1,2,4-triazolium chloride (C.I. 11,055; Basic Red 22), 2-hydroxy-1-[(2-methoxyphenyl)azo)-7-(trimethylammonio)naphthalene chloride (C.I. 12,245; Basic Red 76), di[4-(dimethylamino)phenyl]iminomethane hydrochloride (C.I. 41,000; Basic Yellow 2), 2-[2-((2,4-dimethoxyphenyl)amino)ethenyl]-1,3,3-trimethyl-3H-indol-1-ium chloride (C.I. 48,055; Basic Yellow 11), 3-methyl-1-phenyl-4-[(3-(trimethylammonio)phenyl)azo]pyrazol-5-one chloride (C.I. 12,719; Basic Yellow 57), bis[4-(diethylamino)phenyl]phenylcarbenium hydrogensulfate (1.1) (C.I. 42,040; Basic Green 1), di(4-(dimethylamino)phenyl)phenylmethanol (C.I. 42,000; Basic Green 4), 1-(2-morpholiniumpropylamino)-4-hydroxy-9,10-anthraquinone methylsulfate, 1-[(3-(dimethylpropylaminium)propyl)amino]-4-(methylamino)-9,10-anthraquinone chloride, and direct dyes which contain a heterocycle that comprises at least one quaternary nitrogen atom.

Preferred cationic direct dyes in this context are
(a) cationic triphenylmethane dyes such as, for example, Basic Blue 7, Basic Blue 26, Basic Violet 2, and Basic Violet 14,
(b) aromatic systems that are substituted with a quaternary nitrogen group such as, for example, Basic Yellow 57, Basic Red 76, Basic Blue 99, Basic Brown 16 and Basic Brown 17, and
(c) direct dyes which contain a heterocycle that comprises at least one quaternary nitrogen atom, as recited for example in EP-A2-998 908, to which reference is explicitly made at this juncture, in claims 6 to 11.

Preferred cationic direct dyes of group (c) are, in particular, the following compounds:

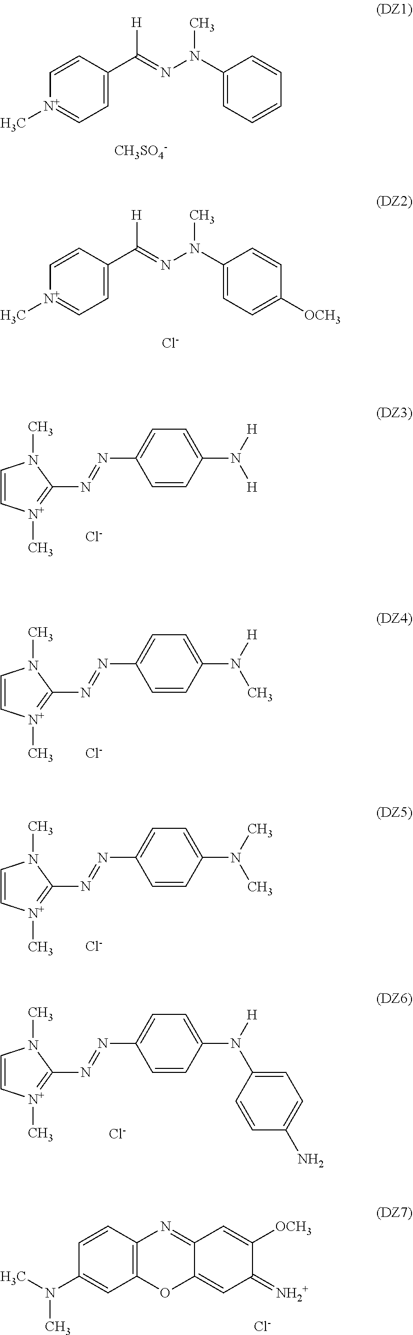

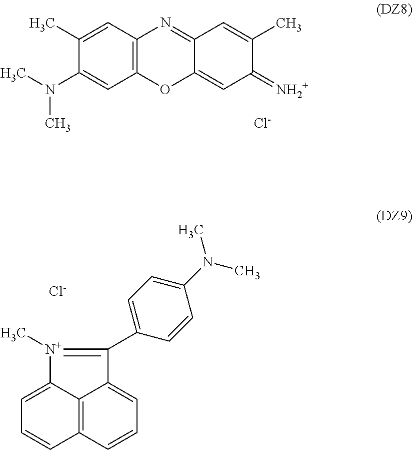

The compounds of formulas (DZ1), (DZ3), and (DZ5), which are also known by the designations Basic Yellow 87, Basic Orange 31, and Basic Red 51, are very particularly preferred cationic direct dyes of group (c).

The cationic direct dyes marketed under the trademark Arianor® are likewise very particularly preferred cationic direct dyes according to the present invention.

The products according to the present invention can furthermore also contain nonionic direct dyes. Nitro dyes, quinone dyes, and neutral azo dyes are particularly suitable.

Suitable blue nitro dyes are, in particular: 1,4-bis[(2-hydroxyethyl)amino]-2-nitrobenzene, 1-(2-hydroxyethyl)amino-2-nitro-4-[di(2-hydroxyethyl)amino]benzene (HC Blue 2), 1-methylamino-4-[methyl-(2,3-dihydroxypropyl)amino]-2-nitrobenzene (HC Blue 6),1-[(2,3-dihydroxypropyl)-amino]-4-[ethyl-(2-hydroxyethyl)amino]-2-nitrobenzene hydrochloride (HC Blue 9), 1-[(2,3-dihydroxypropyl)amino]-4-[methyl-(2-hydroxyethyl)amino]-2-nitrobenzene (HC Blue 10), 4-[di(2-hydroxyethyl)amino]-1-[(2-methoxyethyl)amino]-2-nitrobenzene (HC Blue 11), 4-[ethyl-(2-hydroxyethyl)-amino]-1-[(2-hydroxyethyl)amino]-2-nitrobenzene hydrochloride (HC Blue 12), 2-((4-amino-2-nitrophenyl)amino)-5-dimethylamino benzoic acid (HC Blue 13), 1-amino-3-methyl-4-[(2-hydroxyethyl)amino]-6-nitrobenzene (HC Violet 1), 1-(3-hydroxypropylamino)-4-[di(2-hydroxyethyl)amino]-2-nitrobenzene (HC Violet 2), 1-(2-aminoethylamino)-4-[di(2-hydroxyethyl)amino]-2-nitrobenzene, 4-(Di(2-hydroxyethyl)amino)-2-nitro-1-phenylaminobenzene.

Suitable red nitro dyes are, in particular: 1-Amino-4-[(2-hydroxyethyl)amino]-2-nitrobenzene (HC Red 7), 2-amino-4,6-dinitrophenol (picramic acid) and salts thereof, 1,4-diamino-2-nitrobenzene (C.I. 76,070), 4-amino-2-nitrodiphenylamine (HC Red 1), 1-amino-4-[di(2-hydroxyethyl)amino]2-nitrobenzene hydrochloride (HC Red 13), 1-amino-4-[(2-hydroxyethyl)amino]-5-chloro-2-nitrobenzene, 4-amino-1-[(2-hydroxyethyl)amino]-2-nitrobenzene (HC Red 3), 4-[(2-hydroxyethyl)methylamino)-1-(methylamino)-2-nitrobenzene, 1-amino-4-[(2,3-dihydroxypropyl)amino]-5-methyl-2-nitrobenzene, 1-amino-4-(methylamino)-2-nitrobenzene, 4-amino-2-nitro-1-[(prop-2-en-1-yl)amino]benzene, 4-amino-3-nitrophenol, 4-[(2-hydroxyethyl)-amino]-3-nitrophenol, 4-[(2-nitrophenyl)amino]phenol (HC Orange 1), 1-[(2-aminoethyl)amino]-4-(2-hydroxyethoxy)-2-nitrobenzene (HC Orange 2), 4-(2,3-dihydroxypropoxy)-1-[(2-hydroxyethyl)amino]-2-nitrobenzene (HC Orange 3), 1-amino-5-chloro-4-[(2,3-dihydroxypropyl)amino]-2-nitrobenzene (HC Red 10), 5-chloro-1,4-[di(2,3-dihydroxypropyl)amino]-2-nitrobenzene (HC Red 11), 2-[(2-hydroxyethyl)amino]-4,6-dinitrophenol, 4-ethylamino-3-nitrobenzoic acid, 2-[(4-amino-2-nitrophenyl)amino]benzoic acid, 2-chloro-6-ethylamino-4-nitrophenol, 2-amino-6-chloro-4-nitrophenol, 4-[(3-hydroxypropyl)amino]-3-nitrophenol (HC Red BN), 2,5-diamino-6-nitropyridine, 6-amino-3-[(2-hydroxyethyl)amino]-2-nitropyridine, 3-amino-6-[(2-hydroxyethyl)amino]-2-nitropyridine, 3-amino-6-(ethylamino)-2-nitropyridine, 3-[(2-hydroxyethyl)amino]-6-(methylamino)-2-nitropyridine, 3-amino-6-(methylamino)-2-nitropyridine, 6-(ethylamino)-3-[(2-hydroxyethyl)amino]-2-nitropyridine, 1,2,3,4-tetrahydro-6-nitroquinoxaline, 7-amino-3,4-dihydro-6-nitro-2H-1,4-benzoxazine (HC Red 14).

Suitable yellow nitro dyes are, in particular: 1,2-diamino-4-nitrobenzene (C.I. 76,020), 1-[(2-hydroxyethyl)amino]-2-nitrobenzene (HC Yellow 2), 1-(2-hydroxyethoxy)-2-[(2-hydroxyethyl)amino]-5-nitrobenzene (HC Yellow 4), 1-amino-2-[(2-hydroxyethyl)amino]-5-nitrobenzene (HC Yellow 5), 4-[(2,3-dihydroxypropyl)amino]-3-nitro-1-trifluoromethylbenzene (HC Yellow 6), 2-[di(2-hydroxyethyl)amino]-5-nitrophenol, 2-[(2-hydroxyethyl)amino]-1-methoxy-5-nitrobenzene, 2-amino-3-nitrophenol, 2-amino-4-nitrophenol, 1-amino-2-methyl-6-nitrobenzene, 1-(2-hydroxyethoxy)-3-methylamino-4-nitrobenzene, 2,3-(dihydroxypropoxy)-3-methylamino-4-nitrobenzene, 3-[(2-aminoethyl)amino]-1-methoxy-4-nitrobenzene hydrochloride (HC Yellow 9), 1-chloro-2,4-bis[(2-hydroxyethyl)amino]-5-nitrobenzene (HC Yellow 10), 2-[(2-hydroxyethyl)amino]-5-nitrophenol (HC Yellow 11), 1-[(2′-ureidoethyl)amino]-4-nitrobenzene, 1-amino-4-[(2-aminoethyl)amino]-5-methyl-2-nitrobenzene, 4-[(2-hydroxyethyl)amino]-3-nitro-1-methylbenzene, 1-chloro-4-[(2-hydroxyethyl)amino]-3-nitrobenzene (HC Yellow 12), 4-[(2-hydroxyethyl)amino]-3-nitro-1-trifluoromethylbenzene (HC Yellow 13), 4-[(2-hydroxyethyl)-amino]-3-nitrobenzonitrile (HC Yellow 14), 4-[(2-hydroxyethyl)amino]-3-nitrobenzamide (HC Yellow 15) 3-[(2-hydroxyethyl)amino]-4-methyl-1-nitrobenzene, 4-chloro-3-[(2-hydroxyethyl)amino]-1-nitrobenzene.

Suitable quinone dyes are, in particular; 1,4-di[(2,3-dihydroxypropyl)amino]-9,10-anthraquinone, 1,4-di[(2-hydroxyethyl)amino]-9,10-anthraquinone (C.I. 61,545, Disperse Blue 23), 1-[(2-hydroxyethyl)amino]-4-methylamino-9,10-anthraquinone (C.I. 61,505, Disperse Blue 3), 2-[(2-aminoethyl)amino]-9,10-anthraquinone (HC Orange 5), 1-amino-4-hydroxy-9,10-anthraquinone (C.I. 60,710, Disperse Red 15), 1-hydroxy-4-[(4-methyl-2-sulfophenyl)amino]-9,10-anthraquinone, 7-beta-D-glucopyranosyl-9,10-dihydro-1-methyl-9,10-dioxo-3,5,6,8-tetrahydroxy-2-anthracenecarboxylic acid (C.I. 75,470, Natural Red 4), 1-[(3-aminopropyl)amino]-4-methylamino-9,10-anthraquinone (HC Blue 8), 1-[(3-aminopropyl)amino]-9,10-anthraquinone (HC Red 8), 1,4-diamino-2-methoxy-9,10-anthraquinone (C.I. 62,015, Disperse Red 11, Solvent Violet No. 26), 1,4-dihydroxy-5,8-bis[(2-hydroxyethyl)amino]-9,10-anthraquinone (C.I, 62,500, Disperse Blue 7, Solvent Blue No. 69), 1,4-diamino-9,10-anthraquinone (C.I. 61,100, Disperse Violet 1), 1-amino-4-(methylamino)-9,10-anthraquinone (C.I. 61,105, Disperse Violet 4, Solvent Violet No. 12), 2-hydroxy-3-methoxy-1,4-naphthoquinone, 2,5-dihydroxy-1,4-naphthoquinone, 2-hydroxy-3-methyl-1,4-naphthoquinone, N-{6-[(3-chloro-4-(methylamino)phenyl)imino]-4-methyl-3-oxo-1,4-cyclohexadien-1-yl}urea (HC Red 9), 2-{{4-[di(2-hydroxyethyl)amino]phenyl}amino}-5-[(2-hydroxyethyl)amino]-2,5-cyclohexadiene-1,4-dione (HC Green 1), 5-hydroxy-1,4-naphthoquinone (C.I. 75,500, Natural Brown 7), 2-hydroxy-1,4-naphthoquinone (C.I. 75,480, Natural Orange 6), 1,2-dihydro-2-(1,3-dihydro-3-oxo-2H-indol-2-ylidene)-3H-indol-3-one (C.I. 73,000), 4-{{5-[(2-hydroxyethyl)amino]-1-methyl-1H-pyrazol-4-yl}imino}-4,5-dihydro-5-[(2-hydroxyethyl)-imino]-1-methyl-1H-pyrazole sulfate (1:1), hydrate (1:1).

Suitable neutral azo dyes are, in particular: 1-[di(2-hydroxyethyl)amino]-3-methyl-4-[(4-nitrophenyl)azo]benzene (C.I. 11,210, Disperse Red 17), 1-[di(2-hydroxyethyl)amino]-4-[(4-nitrophenyl)azo]benzene (Disperse Black 9), 4-[(4-aminophenyl)azo]-1-[di(2-hydroxyethyl)amino]-3-methylbenzene (HC Yellow 7), 2,6-diamino-3-[(pyridin-3-yl)azo]pyridine, 2-{[4-(acetylamino)phenyl]azo}-4-methylphenol (C.I. 11,855; Disperse Yellow 3), 4-[(4-nitrophenyl)azo]aniline (C.I. 11,005; Disperse Orange 3).

Preferred nonionic direct dyes are the compounds known under the international designations or trade names HC Yellow 2, HC Yellow 4, HC Yellow 5, HC Yellow 6, HC Yellow 12, HC Orange 1, Disperse Orange 3, HC Red 1, HC Red 3, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9, as well as 1,4-diamino-2-nitrobenzene, 2-amino-4-nitrophenol, 1,4-bis-(2-hydroxyethyl)amino-2-nitrobenzene, 3-nitro-4-(2-hydroxyethyl)aminophenol, 2-(2-hydroxyethyl)amino-4,6-dinitrophenol, 4-[(2-hydroxyethyl)amino]-3-nitro-1-methylbenzene, 1-amino-4-(2-hydroxyethyl)-amino-5-chloro-2-nitrobenzene, 4-amino-3-nitrophenol, 1-(2′-ureidoethyl)amino-4-nitrobenzene, 2-[(4-amino-2-nitrophenyl)amino]benzoic acid, 6-nitro-1,2,3,4-tetrahydroquinoxaline, 2-hydroxy-1,4-naphthoquinone, picramic acid and salts thereof, 2-amino-6-chloro-4-nitrophenol, 4-ethylamino-3-nitrobenzoic acid, and 2-chloro-6-ethylamino-4-nitrophenol.

Depending on the further constituents of the products according to the present invention, the addition of nonionic direct dyes, in particular corresponding nitro dyes, can result in problems in terms of the stability of the dyes or of the respective products. The nonionic direct dyes must therefore be carefully matched to the particular product.

In a preferred embodiment, the products according to the present invention contain no blue nitro dyes at all. Products in which the addition of nitro dyes is entirely omitted are particularly preferred. Products that contain, as direct dyes, exclusively anionic and (if applicable) cationic direct dyes are very particularly preferred.

It is not necessary for the direct dyes to represent uniform compounds in each case. The agents according to the present invention can instead, governed by the manufacturing process for the individual dyes, also contain further components in subordinate quantities, provided they do not disadvantageously influence the color result or do not need to be excluded for other (e.g. toxicological) reasons.

The products according to the present invention can furthermore also contain naturally occurring dyes, for example such as those contained in red henna, neutral henna, black henna, chamomile blossoms, sandalwood, black tea, buckthorn bark, salvia, logwood, madder root, catechu, Spanish cedar, and alkanna root.

In a further embodiment of the present invention, the products can contain at least one precursor of a bioanalogous dye. Those indoles and indolines that comprise at least one hydroxy or amino group, preferably as a substituent on the six-membered ring, are preferred as precursors of bioanalogous dyes. These groups can carry further substituents, e.g. in the form of an etherification or esterification of the hydroxy group or an alkylation of the amino group.

Particularly suitable as precursors of bioanalogous hair dyes are derivatives of 5,6-dihydroxyindoline of formula (NAV I)

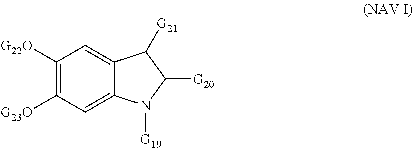

in which, mutually independently,
G₁₉ denotes hydrogen, a C₁ to C₄ alkyl group, or a C₁ to C₄ hydroxyalkyl group,
G₂₀ denotes hydrogen or a —COOH group, such that the —COOH group can also be present as a salt with a physiologically acceptable cation,
G₂₁ denotes hydrogen or a C₁ to C₄ alkyl group,
G₂₂ denotes hydrogen, a C₁ to C₄ alkyl group, or a —CO-G₂₄ group in which G₂₄ denotes a C₁ to C₄ alkyl group, and
G₂₃ denotes one of the groups recited under G₂₂,
   as well as physiologically acceptable salts of these compounds with an organic or inorganic acid.

Particularly preferred derivatives of indoline are 5,6-dihydroxyindoline, N-methyl-5,6-dihydroxyindoline, N-ethyl-5,6-dihydroxyindoline, N-propyl-5,6-dihydroxyindoline, N-butyl-5,6-dihydroxyindoline, 5,6-dihydroxyindoline-2-carboxylic acid, as well as 6-hydroxyindoline, 6-aminoindoline, and 4-aminoindoline.

Particularly to be emphasized within this group are N-methyl-5,6-dihydroxyindoline, N-ethyl-5,6-dihydroxyindoline, N-propyl-5,6-dihydroxyindoline, N-butyl-5,6-dihydroxyindoline, and in particular 5,6-dihydroxyindoline.

Also outstandingly suitable as precursors of bioanalogous hair dyes are derivatives of 5,6-dihydroxyindole of formula (NAV II)

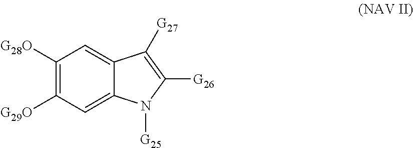

in which, mutually independently:
G₂₅ denotes hydrogen, a C₁ to C₄ alkyl group, or a C₁ to C₄ hydroxyalkyl group,
G₂₆ denotes hydrogen or a —COOH group, such that the —COOH group can also be present as a salt with a physiologically acceptable cation,
G₂₇ denotes hydrogen or a C₁ to C₄ alkyl group,
G₂₈ denotes hydrogen, a C₁ to C₄ alkyl group, or a —CO-G₃₀ group in which G₃₀ denotes a C₁ to C₄ alkyl group, and
G₂₉ denotes one of the groups recited under G₂₈,
as well as physiologically acceptable salts of these compounds with an organic or inorganic acid.

Particularly preferred derivatives of indole are 5,6-dihydroxyindole, N-methyl-5,6-dihydroxyindole, N-ethyl-5,6-dihydroxyindole, N-propyl-5,6-dihydroxyindole, N-butyl-5,6-dihydroxyindole, 5,6-dihydroxyindole-2-carboxylic acid, 6-hydroxyindole, 6-aminoindole, and 4-aminoindole.

To be emphasized within this group are N-methyl-5,6-dihydroxyindole, N-ethyl-5,6-dihydroxyindole, N-propyl-5,6-dihydroxyindole, N-butyl-5,6-dihydroxyindole, and in particular 5,6-dihydroxyindole.

The indoline and indole derivatives can be used in the products according to the present invention both as free bases and in the form of physiologically acceptable salts thereof with inorganic or organic acids, e.g. the hydrochlorides, sulfates, and hydrobromides. The indole or indoline derivatives are contained therein usually in quantities from 0.05 to 10 wt %, by preference 0.2 to 5 wt %.

in a further embodiment, it may be preferred according to the present invention to use the indoline or indole derivative in coloring agents in combination with at least one amino acid or one oligopeptide. The amino acid is advantageously an α-amino acid; very particularly preferred α-amino acids are arginine, ornithine, lysine, serine, and histidine, in particular arginine.

The film-forming and/or setting polymers and the dyes are incorporated into a cosmetically acceptable carrier. This preferably involves an aqueous, alcoholic, or aqueous/alcoholic medium having by preference at least 10 wt % water, based on the entire foam-type or foamable composition. The alcohols contained can be, in particular, the lower alcohols having 1 to 4 carbon atoms usually used for cosmetic purposes, for example ethanol and isopropanol. Particularly preferably, the cosmetically acceptable carrier is water.

Organic solvents, or a mixture of solvents having a boiling point under 400° C., can be contained as additional co-solvents in a quantity from 0.1 to 15 weight percent, preferably from 1 to 10 weight percent, based on the entire foam-type or foamable composition. Unbranched or branched hydrocarbons such as pentane, hexane, isopentane, and cyclic hydrocarbons such as cyclopentane and cyclohexane, are particularly suitable as additional co-solvents. Further particularly preferred water-soluble solvents are glycerol, ethylene glycol, and propylene glycol, in a quantity of up to 30 wt % based on the entire preparation.

The products according to the present invention are aerosol-foam products or pump-foam products based on a foam-type or foamable composition. It may be necessary to add to the compositions ingredients that promote foam formation or that stabilize foam once it has been formed. Surfactants and/or emulsifiers are particularly suitable for this.

Cationic surfactants, which in addition to foam-forming and foam-stabilizing properties also exhibit a care-providing effect, are particularly suitable.

Cationic surfactants of the quaternary ammonium compound, esterquat, and amidoamine types are preferred according to the present invention.

Esterquats are known substances that contain both at least one ester function and at least one quaternary ammonium group as a structural element. Preferred esterquats are quaternized ester salts of fatty acids with triethanolamine, quaternized ester salts of fatty acids with diethanolalkylamines, and quaternized ester salts of fatty acids with 1,2-dihydroxypropyldialkylamines. Such products are marketed, for example, under the trademarks Stepantex®, Dehyquart®, and Armocare®. Examples of such esterquats are the products Armocare® VGH-70—an N,N-bis(2-palmitoyloxyethyl)dimethylammonium chloride—as well as Dehyquart® F-75, Dehyquart® C-4046, Dehyquart® L-80, Dehyquart® AU-35.

The alkylamidoamines are usually produced by amidation of natural or synthetic fatty acids and fatty acid cuts with dialkylaminoamines. One compound from this group of substances that is particularly suitable according to the present invention is the stearamidopropyldimethylamine available commercially under the designation Tegoamid® S 18.

Particularly preferred cationic surfactants are the quaternary ammonium compounds. The foam-type or foamable composition therefore further contains, by preference, at least one quaternary ammonium compound.

Suitable quaternary ammonium compounds are, for example, ammonium halides, in particular chlorides and bromides, such as alkyltrimethylammonium chlorides, dialkyldimethylammonium chlorides, and trialkylmethylammonium chlorides, e.g. cetyltrimethylammonium chloride, stearyltrimethylammonium chloride, distearyldimethylammonium chloride, lauryldimethylammonium chloride, lauryldimethylbenzylammonium chloride, and tricetylmethylammonium chloride, as well as the imidazolium compounds known under the INCI names Quaternium-27 and Quaternium-83. The long alkyl chains of the aforesaid surfactants preferably comprise 10 to 18 carbon atoms.

Particularly preferably, the foam-type or foamable composition contains at least one quaternary ammonium compound of formula (Q-I)

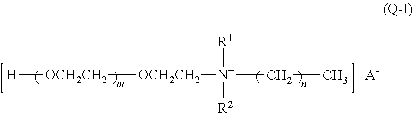

in which
R¹ and R², mutually independently in each case, denote an optionally hydroxy-substituted C₁ to C₄ alkyl group,
m denotes an integer from 0 to 20,
n denotes an integer from 0 to 20, and
A denotes a monovalent anion.

Very particularly preferably, the foam-type or foamable composition contains at least one quaternary ammonium compound of formula (Q-I), in which R¹ and R² denote methyl, m denotes 0, n denotes an integer from 9 to 17, and A⁻ denotes a monovalent anion.

The cationic surfactants are contained in the products according to the present invention preferably in quantities from 0.05 to 10 wt %, based on the entire foam-type or foamable composition. Quantities from 0.1 to 5 wt % are particularly preferred.

In addition to or instead of the cationic surfactants, the foam-type or foamable compositions can contain further surfactants or emulsifiers, both anionic as well as ampholytic and nonionic surfactants, and all types of known emulsifiers, being suitable in principle. The group of the ampholytic or amphoteric surfactants encompasses zwitterionic surfactants and ampholytes. The surfactants can already have an emulsifying effect.

The foam-type or foamable compositions by preference, however, contain at least one cationic surfactant. Particularly preferably, the foam-type or foamable compositions contain exclusively cationic surfactants.

All anionic surface-active substances suitable for utilization on the human body are suitable in principle as anionic surfactants. These substances are characterized by a water-solubility-creating anionic group such as, for example, a carboxylate, sulfate, sulfonate, or phosphate group, and a lipophilic alkyl group having approximately 8 to 30 carbon atoms. Glycol or polyglycol ether groups, ester, ether, and amide groups, and hydroxyl groups can additionally be contained in the molecule. Examples of suitable anionic surfactants are, in each case in the form of the sodium, potassium, and ammonium as well as mono-, di-, and trialkanolammonium salts having 2 to 4 carbon atoms in the alkanol group:
linear and branched fatty acids having 8 to 30 carbon atoms (soaps);
ethercarboxylic acids of the formula R—O—(CH₂—CH₂O)ₓ—CH₂—COOH, in which R is a linear alkyl group having 8 to 30 carbon atoms and x=0 or is 1 to 16;
acylsarcosides having 8 to 24 carbon atoms in the acyl group;
acyltaurides having 8 to 24 carbon atoms in the acyl group;
acylisethionates having 8 to 24 carbon atoms in the acyl group;
sulfosuccinic acid mono- and -dialkyl esters having 8 to 24 carbon atoms in the alkyl group, and sulfosuccinic acid monoalkylpolyoxyethyl esters having 8 to 24 carbon atoms in the alkyl group and 1 to 6 oxyethyl groups;
linear alkanesulfonates having 8 to 24 carbon atoms;
linear alpha-olefinsulfonates having 8 to 24 carbon atoms;
alpha-sulfofatty acid methyl esters of fatty acids having 8 to 30 carbon atoms;
alkyl sulfates and alkylpolyglycol ether sulfates of the formula R—O(CH₂—CH₂O)ₓ—OSO₃H, in which R is a preferably linear alkyl group having 8 to 30 carbon atoms and x=0 or is 1 to 12;
mixtures of surface-active hydroxysulfonates;
sulfated hydroxyalkylpolyethylene and/or hydroxyalkylenepropylene glycol ethers;
sulfonates of unsaturated fatty acids having 8 to 24 carbon atoms and 1 to 6 double bonds;
esters of tartaric acid and citric acid with alcohols, which represent addition products of approximately 2 to 15 molecules ethylene oxide and/or propylene oxide with fatty alcohols having 8 to 22 carbon atoms.
alkyl and/or alkenyl ether phosphates of formula (E1-I)

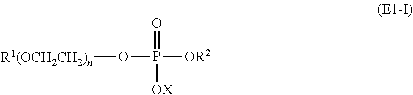

in which R¹ preferably denotes an aliphatic hydrocarbon radical having 8 to 30 carbon atoms, R² denotes hydrogen, a (CH₂CH₂O)ₙR¹ radical, or X, n denotes numbers from 1 to 10, and X denotes hydrogen, an alkali or alkaline-earth metal, or NR³R⁴R⁵R⁶, where R³ to R⁶, mutually independently, denote hydrogen or a C₁ to C₄ hydrocarbon radical;
sulfated fatty acid alkylene glycol esters of the formula (E1-II)

R⁷CO(AlkO)ₙSO₃M (E1-II)

in which R⁷CO— denotes a linear or branched, aliphatic, saturated and/or unsaturated acyl radical having 6 to 22 carbon atoms, Alk denotes CH₂CH₂, CHCH₃CH₂, and/or CH₂CHCH₃, n denotes numbers from 0.5 to 5, and M denotes a cation, as described in German Application 197 36 906;
monoglyceride sulfates and monoglyceride ether sulfates of formula (E1-III)

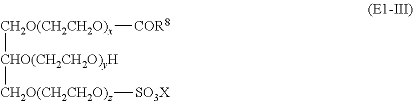

in which R⁸CO denotes a linear or branched acyl radical having 6 to 22 carbon atoms, x, y, and z in total denote 0 or numbers from 1 to 30, by preference 2 to 10, and X denotes an alkali or alkaline-earth metal, Typical examples of monoglyceride (ether) sulfates suitable for purposes of the invention are the reaction products of lauric acid monoglyceride, coconut fatty acid monoglyceride, palmitic acid monoglyceride, stearic acid monoglyceride, oleic acid monoglyceride, and tallow fatty acid monoglyceride, and their ethylene oxide adducts with sulfur trioxide or chlorosulfonic acid in the form of their sodium salts. Monoglyceride sulfates of formula (E1-III) in which R⁸CO denotes a linear acyl radical having 8 to 18 carbon atoms are preferably used;
amide ethercarboxylic acids;
condensation products of C₈ to C₃₀ fatty alcohols with protein hydrolysates and/or amino acids and their derivatives, known to one skilled in the art as protein fatty acid condensates, such as, for example, Lamepon® grades, Gluadin® grades, Hostapon® KCG, or the Amisoft® grades.

Preferred anionic surfactants are alkyl sulfates, alkyl polyglycol ether sulfates, and ethercarboxylic acids having 10 to 18 carbon atoms in the alkyl group and up to 12 glycol ether groups in the molecule, sulfosuccinic acid mono- and dialkyl esters having 8 to 18 carbon atoms in the alkyl group, and sulfosuccinic acid monoalkylpolyoxyethyl esters having 8 to 18 carbon atoms in the alkyl group and 1 to 6 oxyethyl groups, monoglycerol disulfates, alkyl and alkenyl ether phosphates, and protein fatty acid condensates.

“Zwitterionic surfactants” refers to those surface-active compounds that contain in the molecule at least one quaternary ammonium group and at least one —COO⁽⁻⁾ or —SO₃⁽⁻⁾ group. Particularly suitable zwitterionic surfactants are the so-called betaines, such as the N-alkyl-N,N-dimethylammonium glycinates, for example cocalkyldimethylammonium glycinate, N-acylaminopropyl-N,N-dimethylammonium glycinates, for example cocacylaminopropyldimethylammonium glycinate, and 2-alkyl-3-carboxymethyl-3-hydroxyethylimidazolines, having in each case 8 to 18 carbon atoms in the alkyl or acyl group, as well as cocacylaminoethylhydroxyethylcarboxymethyl glycinate. A preferred zwitterionic surfactant is the fatty acid amide derivative known by the INCI name Cocamidopropyl Betaine.

“Ampholytes” are understood as those surface-active compounds that contain in the molecule, in addition to a C₈ to C₂₄ alkyl or acyl group, at least one free amino group and at least one —COOH or —SO₃H group, and are capable of forming internal salts. Examples of suitable ampholytes are N-alkyl-glycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids, and alkylaminoacetic acids, having in each case 8 to 24 carbon atoms in the alkyl group. Particularly preferred ampholytes are N-cocalkylaminopropionate, cocacylaminoethylaminopropionate, and C₁₂to C₁₈ acylsarcosine.

Nonionic surfactants contain as a hydrophilic group, for example, a polyol group, a polyalkylene glycol ether group, or a combination of a polyol and polyglycol ether group. Such compounds are, for example:
addition products of 2 to 50 mol ethylene oxide and/or 1 to 5 mol propylene oxide with linear and branched fatty alcohols having 8 to 30 carbon atoms, with fatty acids having 8 to 30 carbon atoms, and with alkylphenols having 8 to 15 carbon atoms in the alkyl group;
addition products, end-capped with a methyl or C₂ to C₆ alkyl group, of 2 to 50 mol ethylene oxide and/or 1 to 5 mol propylene oxide with linear and branched fatty alcohols having 8 to 30 carbon atoms, with fatty acids having 8 to 30 carbon atoms, and with alkylphenols having 8 to 15 carbon atoms in the alkyl group, such as, for example, the grades obtainable under the marketing designations Dehydol® LS, Dehydol® LT (Cognis);
C₁₂ to C₃₀ fatty acid mono- and diesters of addition products of 1 to 30 mol ethylene oxide with glycerol;
addition products of 5 to 60 mol ethylene oxide with castor oil and hardened castor oil;
polyol fatty acid esters such as, for example, the commercial product Hydagen® HSP (Cognis), or Sovermol grades (Cognis);
alkoxylated triglycerides;
alkoxylated fatty acid alkyl esters of formula (E4-I).

R¹CO—(OCH₂OHR²)_{w}OR³ (E4-I),

in which R¹CO denotes a linear or branched, saturated and/or unsaturated acyl radical having 6 to 22 carbon atoms, R² denotes hydrogen or methyl, R³ denotes linear or branched alkyl radicals having 1 to 4 carbon atoms, and w denotes numbers from 1 to 20;
amine oxides;
hydroxy mixed ethers, such as those described e.g. in German Application 197 38 866;
sorbitan fatty acid esters and addition products of ethylene oxide with sorbitan fatty acid esters, for example the polysorbates;
sugar fatty acid esters and addition products of ethylene oxide with sugar fatty acid esters;
addition products of ethylene oxide with fatty acid alkanolamides and fatty amines;
sugar surfactants of the alkyl and alkenyl oligoglycoside types, according to formula (E4-II)

R⁴O-[G]ₚ (E4-II),

in which R⁴ denotes an alkyl or alkenyl radical having 4 to 22 carbon atoms, G denotes a sugar radical having 5 or 6 carbon atoms, and p denotes numbers from 1 to 10. They can be obtained in accordance with the relevant methods of preparative organic chemistry.

The alkyl and alkenyl oligoglycosides can be derived from aldoses or ketoses having 5 or 6 carbon atoms, preferably from glucose. The preferred alkyl and/or alkenyl oligoglycosides are thus alkyl and/or alkenyl oligoglucosides. The index number p in the general formula (E4-II) indicates the degree of oligomerization (DP), i.e. the distribution of mono- and oligoglycosides, and denotes a number between 1 and 10. Whereas p in the individual molecule must always be integral, and here can principally assume the values p=1 to 6, the value p for a specific alkyl oligoglycoside is an analytically ascertained calculated value that usually represents a fractional number. Alkyl and/or alkenyl oligoglycosides having an average degree of oligomerization p from 1.1 to 3.0 are preferably used. In terms of applications engineering, those alkyl and/or alkenyl oligoglycosides whose degree of oligomerization is less than 1.7, and in particular between 1.2 and 1.4, are preferred. The alkyl or alkenyl radical R⁴ can be derived from primary alcohols having 4 to 11, preferably 8 to 10 carbon atoms. Typical examples are butanol, hexanol, octanol, decanol, and undecyl alcohol as well as industrial mixtures thereof, such as those obtained, for example, upon hydrogenation of industrial fatty acid methyl esters or in the course of the hydrogenation of aldehydes from Roelen oxosynthesis. Preferred are alkyl oligoglucosides of chain length C₈ to C₁₀ (DP=1 to 3), which occur as the first runnings upon distillational separation of industrial C₈ to C₁₈ coconut oil alcohol and can be contaminated with a proportion of less than 6 wt % C₁₂ alcohol, and alkyl oligoglucosides based on industrial C_{9/11} oxoalcohols (DP=1 to 3). The alkyl or alkenyl radical R¹⁵ can furthermore also be derived from primary alcohols having 12 to 22, preferably 12 to 14 carbon atoms. Typical examples are lauryl alcohol, myristyl alcohol, cetyl alcohol, palmoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, brassidyl alcohol and industrial mixtures thereof, which can be obtained as described above. Alkyl oligoglucosides based on hardened C_{12/14} cocalcohol having a DP of 1 to 3 are preferred.
sugar surfactants of the type of the fatty acid N-alkylpolyhydroxyalkylamides, a nonionic surfactant of the formula (E4-III)

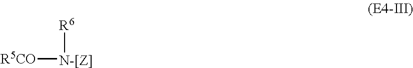

in which R⁵CO denotes an aliphatic acyl radical having 6 to 22 carbon atoms, R⁶ denotes hydrogen, an alkyl or hydroxyalkyl radical having 1 to 4 carbon atoms, and [Z] denotes a linear or branched polyhydroxyalkyl radical having 3 to 12 carbon atoms and 3 to 10 hydroxyl groups. The fatty acid N-alkylpolyhydroxyalkylamides are known substances that can usually be obtained by reductive amination of a reducing sugar with ammonia, an alkylamine, or an alkanolamine, and subsequent acylation with a fatty acid, a fatty acid alkyl ester, or a fatty acid chloride. With regard to the method for their manufacture, reference may be made to U.S. Pat. No. 1,985,424, U.S. Pat. No. 2,016,962 and U.S. Pat. No. 2,703,798, and to International Patent Application WO 92/06984. The fatty acid N-alkylpolyhydroxyalkylamides are preferably derived from reducing sugars having 5 or 6 carbon atoms, in particular from glucose. The preferred fatty acid N-alkylpolyhydroxyalkylamides therefore represent fatty acid N-alkylglucamides such as those reproduced by formula (E4-IV):

R⁷CO—NR⁸—CH₂—(CHOH)₄—CH₂OH (E4-IV).

It is preferable to use, as fatty acid N-alkylpolyhydroxyalkylamides, glucamides of formula (E4-IV) in which R⁸ denotes hydrogen or an alkyl group and R⁷CO denotes the acyl radical of hexanoic acid, octanoic acid, decanoic acid, lauric acid, myristic acid, palmitic acid, palmoleic acid, stearic acid, isostearic acid, oleic acid, elaidic acid, petroselinic acid, linoleic acid, linolenic acid, arachidic acid, gadoleic acid, behenic acid, erucic acid, or industrial mixtures thereof Particularly preferred are fatty acid N-alkylglucamides of formula (E4-IV) that are obtained by reductive amination of glucose with methylamine and subsequent acylation with lauric acid or C_{12/14} coconut fatty acid, or a corresponding derivative. The polyhydroxyalkylamides can furthermore also be derived from maltose and palatinose.

The alkylene oxide addition products with saturated linear fatty alcohols and fatty acids, having respectively 2 to 30 mol ethylene oxide per mol fatty alcohol or fatty acid, have proven to be preferred nonionic surfactants. Preparations having outstanding properties are likewise obtained if they contain, as nonionic surfactants, fatty acid esters of ethoxylated glycerol.

These compounds are characterized by the following parameters: The alkyl radical R contains 6 to 22 carbon atoms and can be both linear and branched Primary linear aliphatic radicals, and aliphatic radicals methyl-branched in the 2-position, are preferred. Such alkyl radicals are, for example, 1-octyl, 1-decyl, 1-lauryl, 1-myristyl, 1-cetyl, and 1-stearyl. 1-Octyl, 1-decyl, 1-lauryl, and 1-myristyl are particularly preferred. When so-called “oxo alcohols” are used as the initial materials, compounds having an odd number of carbon atoms in the alkyl chain predominate.

The sugar surfactants can also be contained as nonionic surfactants. They are contained preferably in quantities from 0.1 to 20 wt %, based on the entire respective preparation. Quantities from 0.5 to 15 wt % are preferred, and quantities from 0.5 to 7.5 wt % are very particularly preferred.

The compounds having alkyl groups used as a surfactant can in each case be uniform substances. It is generally preferred, however, to proceed from natural vegetable or animal raw materials when producing these substances, so that substance mixtures having different alkyl chain lengths, as a function of the particular material, are obtained.

In the surfactants that represent addition products of ethylene oxide and/or propylene oxide with fatty alcohols, or derivatives of such addition products, both products having a “normal” homolog distribution and those having a restricted homolog distribution can be used. A “normal” homolog distribution is understood as mixtures of homologs that are obtained when reacting fatty alcohol and alkylene oxide using alkali metals, alkali-metal hydroxides, or alkali-metal alcoholates as catalysts. Restricted homolog distributions, on the other hand, are obtained when, for example, hydrotalcites, alkaline-earth metal salts of ethercarboxylic acids, or alkaline-earth metal oxides, hydroxides or alcoholates are used as catalysts. The use of products having a restricted homolog distribution may be preferred.

The further surfactants are used as a rule in quantities from 0.1 to 45 wt %, preferably 0.5 to 30 wt %, and very particularly preferably from 0.5 to 25 wt %, based on the respective entire composition.

The foam-type or foamable compositions can furthermore contain at least one emulsifier. Emulsifiers cause the formation, at the phase interface, of water- or oil-stable adsorption layers that prevent the dispersed droplets from coalescing and thereby stabilize the emulsion. Emulsifiers are therefore, like surfactants, constructed from a hydrophobic and a hydrophilic molecule part. Hydrophilic emulsifiers preferentially form O/W emulsions, and hydrophobic emulsifiers preferentially form W/O emulsions. Selection of these emulsifying surfactants or emulsifiers is based on the substances to be dispersed and the respective external phase, and on the fineness of the emulsion particles. Emulsifiers usable according to the present invention are, for example:
addition products of 4 to 100 mol ethylene oxide and/or 1 to 5 mol propylene oxide with linear fatty alcohols having 8 to 22 carbon atoms, with fatty acids having 12 to 22 carbon atoms, and with alkylphenols having 8 to 15 carbon atoms in the alkyl group;
C₁₂ to C₂₂ fatty acid mono- and diesters of addition products of 1 to 30 mol ethylene oxide with polyols having 3 to 6 carbon atoms, in particular with glycerol;
addition products of ethylene oxide and polyglycerol with methyl glucoside fatty acid esters, fatty acid alkanolamides, and fatty acid glucamides;
C₈ to C₂₂ alkyl mono- and oligoglycosides and ethoxylated analogs thereof, degrees of oligomerization from 1.1 to 5, in particular 1.2 to 2.0, and glucose as the sugar component, being preferred;
mixtures of alkyl(oligo)glucosides and fatty alcohols, for example the commercially available product Montanov® 68;
addition products of 5 to 60 mol ethylene oxide with castor oil and hardened castor oil;
partial esters of polyols having 3 to 6 carbon atoms with saturated fatty acids having 8 to 22 carbon atoms;
Sterols. “Sterols” are understood as a group of steroids that carry a hydroxyl group on the third carbon atom of the steroid structure and are isolated both from animal tissue (zoosterols) and from vegetable fats (phytosterols). Examples of zoosterols are cholesterol and lanosterol. Examples of suitable phytosterols are ergosterol, stigmasterol, and sitosterol. Sterols called “mycosterols” are also isolated from fungi and yeasts.
Phospholipids. These are understood as principally the glucose phospholipids, which are obtained e.g. as lecithins or phosphatidylcholines from, for example, egg yolk or plant seeds (e.g. soybeans).
fatty acid esters of sugars and sugar alcohols, such as sorbitol;
polyglycerols and polyglycerol derivatives such as, for example, polyglycerol poly-12-hydroxystearate (commercial product Dehymuls® PGPH).
linear and branched fatty acids having 8 to 30 carbon atoms, and their Na, K, ammonium, Ca, Mg, and Zn salts.

The emulsifiers are used preferably in quantities from 0.1 to 25 wt %, in particular 0.1 to 3 wt %, based on the entire respective composition.

Nonionogenic emulsifiers having an HLB value from 8 to 18, according to the definitions set forth in the Römpp-Lexikon Chemie [Römpp chemical dictionary] (J. Falbe, M. Regitz, eds.), 10th edition, Georg Thieme Verlag Stuttgart, New York (1997), page 1764, are preferred Nonionogenic emulsifiers having an HLB value from 10 to 16 are particularly preferred according to the present invention.

The products according to the present invention preferably exhibit a pH of less than 7 The pH range between 5 and 6.5 is particularly preferred. The indications regarding pH refer, for purposes of this document, to the pH at 25° C. unless otherwise noted.

If necessary, usual alkalizing agents or agents for acidifying the composition can be added to the products according to the present invention in order to establish the desired pH.

The products according to the present invention can furthermore contain all further adjuvants, additives, and active substances usual for hair treatment agents.

It has proven to be particularly advantageous if the product according to the present invention furthermore contains at least one care-providing substance. As already stated above, this can refer to a cationic surfactant, which on the one hand exhibits a care-providing effect and additionally can promote foam formation and stabilize the foam that is produced. Cationic surfactants are therefore the preferred care-providing substances.

Protein hydrolysates and/or protein hydrolysate derivatives are also suitable as care-providing substances.

Protein hydrolysates are product mixtures obtained by the acid-, base-, or enzyme-catalyzed breakdown of proteins. The term “protein hydrolysates” is also understood according to the present invention to mean total hydrolysates as well as individual amino acids and their derivatives, as well as mixtures of different amino acids. Polymers constructed from amino acids and amino-acid derivatives are also understood according to the present invention under the term “protein hydrolysates”. Included among the latter are, for example, polyalanine, polyasparagine, polyserine, etc. Further examples of compounds usable according to the present invention are L-alanyl-L-proline, polyglycine, glycyl-L-glutamine, or D/L-methionine-S-methylsulfonium chloride. β-Amino acids and their derivatives, such as β-alanine, anthranilic acid, or hippuric acid, can of course also be used according to the present invention. The molecular weight of the protein hydrolysates usable according to the present invention is between 75 (the molecular weight of glycine) and 200,000; the molecular weight is preferably 75 to 50,000 dalton, and very particularly preferably 75 to 20,000 dalton.

According to the present invention, protein hydrolysates of both plant and animal origin, or of marine or synthetic origin, can be used.

Animal protein hydrolysates are, for example, hydrolysates of elastin, collagen, keratin, silk, and milk protein, which can also be present in the form of salts. Such products are marketed, for example, under the trademarks Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex), Sericin (Pentapharm), and Kerasol® (Croda).

The use of silk protein hydrolysates is of particular interest. “Silk” is understood as the fibers of the cocoon of the mulberry silkworm (Bombyx mori L.). The raw silk fiber is made up of a double thread of fibroin. Sericin serves as a glue substance holding this double thread together. Silk is made up of 70 to 80 wt % fibroin, 19 to 28 wt % sericin, 0.5 to 1 wt % fat, and 0.5 to 1 wt % coloring agents and mineral constituents.

The essential constituents of sericin are approximately 46 wt % hydroxyamino acids. Sericin is made up of a group of 5 to 6 proteins. The essential amino acids of sericin are serine (Ser, 37 wt %), aspartate (Asp, 26 wt %), glycine (Gly, 17 wt %), alanine (Ala), leucine (Leu), and tyrosine (Tyr).

Water-insoluble fibroin is included among the scleroproteins having a long-chain molecular structure. The principal constituents of fibroin are glycine (44 wt %), alanine (26 wt %), and tyrosine (13 wt %). A further essential structural feature of fibroin is the hexapeptide sequence Ser-Gly-Ala-Gly-Ala-Gly.

It is technically simple to separate the two silk proteins from one another. It is therefore not surprising that both sericin and fibroin are known, each individually, as raw materials for use in cosmetic products. Protein hydrolysates and derivatives based on the respective individual silk proteins are also known raw materials in cosmetic agents. For example, sericin as such is marketed by Pentapharm Ltd. as a commercial product with the designation Sericin Code 303-02. Fibroin is offered far more frequently on the market as a protein hydrolysate, at various molecular weights. These hydrolysates are marketed in particular as “silk hydrolysates.” Hydrolyzed fibroin having average molecular weights between 350 and 1000 is marketed, for example, under the commercial designation Promois® Silk. DE 31 39 438 A1 also describes colloidal fibroin solutions as an additive in cosmetic agents.

The positive properties of the silk protein derivatives from sericin and fibroin, each considered individually, are known in the literature. For example, the sales brochure of the Pentapharm company describes the cosmetic effects of sericin on the skin as irritation-soothing, hydrating, and film-forming. The effect of a fibroin derivative is described, for example in DE 31 39 438 A1, as providing care to and revival of the hair. According to DE 102 40 757 A1, with the simultaneous use of sericin and fibroin, or derivatives and/or hydrolysates thereof, it is furthermore possible to achieve a synergistic increase in the positive effects of the silk proteins and their derivatives.

It is therefore preferred to use, as a silk protein hydrolysate, an active-substance complex (A) comprising the active substance (A1) selected from sericin, sericin hydrolysates, and/or derivatives thereof, as well as mixtures thereof, and an active substance (A2) selected from fibroin and/or fibroin hydrolysates and/or derivatives thereof and/or mixtures thereof.

The active-substance complex (A) significantly improves, in synergistic fashion, the essential internal and external structural features presented above, and both the strength and elasticity of human hairs.

The following can be used as active substances (A1) in the active-substance complex (A):
native sericin;
hydrolyzed and/or further derivatized sericin, for example commercial products having the INCI names Sericin, Hydrolyzed Sericin, or Hydrolyzed Silk;
a mixture of the amino acids serine, aspartate, and glycine and/or the methyl, propyl, isopropyl, butyl, isobutyl esters thereof, the salts thereof such as, for example, hydrochlorides, sulfates, acetates, citrates, tartrates, such that the serine and/or derivatives thereof are contained in this mixture at 20 to 60 wt %, the aspartate and/or derivatives thereof at 10 to 40 wt %, and the glycine and/or derivatives thereof at 5 to 30 wt %, with the stipulation that the quantities of these amino acids and/or derivatives thereof by preference add up to 100 wt %; and
mixtures thereof.

The following can be used as active substances (A2) in the active-substance complex (A):
natural fibroin converted into a soluble form;
hydrolyzed and/or further derivatized fibroin, especially partly hydrolyzed fibroin, which contains as a principal constituent the amino acid sequence

Ser-Gly-Ala-Gly-Ala-Gly;

the amino acid sequence Ser-Gly-Ala-Gly-Ala-Gly;
a mixture of the amino acids glycine, alanine, and tyrosine and/or the methyl, propyl, isopropyl, butyl, isobutyl esters thereof, the salts thereof such as, for example, hydrochlorides, sulfates, acetates, citrates, tartrates, such that the glycine and/or derivatives thereof is contained in this mixture in quantities from 20 to 60 wt %, the alanine and derivatives thereof in quantities from 10 to 40 wt %, and the tyrosine and derivatives thereof in quantities from 0 to 25 wt %, with the stipulation that the quantities of these amino acids and/or derivatives thereof by preference add up to 100 wt %; and
mixtures thereof.

Particularly good care-providing properties can be achieved if one of the two active-substance components of the active-substance complex (A) is used in the natural or, if need be, solubilized form. It is also possible to utilize a mixture of several active substances (A1) and/or (A2).

It may be preferred to use the two active substances (A1) and (A2) in the products according to the present invention at a ratio from 10:90 to 70:30, in particular 15:85 to 50:50, and very particularly 20:80 to 40:60, based on their respective active-substance contents.

The derivatives of the hydrolysates of sericin and fibroin encompass both anionic and cationized protein hydrolysates. The protein hydrolysates of sericin and fibroin, and the derivatives manufactured therefrom, can be obtained from the corresponding proteins by way of a chemical, in particular alkaline or acid, hydrolysis, by an enzymatic hydrolysis, and/or by a combination of the two types of hydrolysis. The hydrolysis of proteins generally yields a protein hydrolysate having a molecular weight distribution from approximately 100 daltons to several thousand daltons. Those protein hydrolysates of sericin and fibroin and/or derivatives thereof whose underlying protein fraction has a molecular weight from 100 to 25,000 daltons, preferably 250 to 10,000 daltons, are preferred Quaternized amino acids and mixtures thereof are also to be understood as cationic protein hydrolysates of sericin and fibroin. The quaternization of protein hydrolysates or amino acids is often carried out by means of quaternary ammonium salts such as, for example, N,N-dimethyl-N-(n-alkyl)-N-(2-hydroxy-3-chloro-n-propyl)ammonium halides. The cationic protein hydrolysates can moreover be even further derivatized. Typical examples that may be mentioned of cationic protein hydrolysates and derivatives usable according to the present invention are the following products listed under the INCI names in the “International Cosmetic Ingredient Dictionary and Handbook” (seventh edition 1997, The Cosmetic, Toiletry, and Fragrance Association, 1101 17^{th} Street, N.W., Suite 300, Washington, DC 20036-4702) and available commercially: Cocodimonium Hydroxypropyl Hydrolyzed Silk, Cocodimonium Hydroxypropyl Silk Amino Acids, Hydroxypropyltrimonium Hydrolyzed Silk, Lauryldimonium Hydroxypropyl Hydrolyzed Silk, Steardimonium Hydroxypropyl Hydrolyzed Silk, Quaternium-79 Hydrolyzed Silk. Typical examples that may be mentioned of the anionic protein hydrolysates and derivatives according to the present invention are the following products listed under the INCI names in the “International Cosmetic Ingredient Dictionary and Handbook” (seventh edition 1997, The Cosmetic, Toiletry, and Fragrance Association, 1101 17th Street, N.W., Suite 300, Washington, DC 20036-4702) and commercially available: Potassium Cocoyl Hydrolyzed Silk, Sodium Lauroyl Hydrolyzed Silk, or Sodium Stearoyl Hydrolyzed Silk. Lastly, the following products obtainable commercially under their INCI names may be mentioned as typical examples of the derivatives of sericin and fibroin usable according to the present invention. Ethyl Ester of Hydrolyzed Silk, and Hydrolyzed Silk PG-Propyl Methylsilanediol. Also usable according to the present invention, although not unconditionally preferred, are the commercially obtainable products having the INCI names Palmitoyl Oligopeptide, Palmitoyl Pentapeptide-3, Palmitoyl Pentapeptide-2, Acetyl Hexapeptide-1, Acetyl Hexapeptide-3, Copper Tripeptide-1, Hexapeptide-1, Hexapeptide-2, and MEA-Hydrolyzed Silk.

The effect of active substance complex (A) can be further enhanced by the addition of fatty substances. Fatty substances are to be understood as fatty acids, fatty alcohols, natural and synthetic waxes that can be present both in solid form and in liquid form in aqueous dispersion, and natural and synthetic cosmetic oil components.

Protein hydrolysates of vegetable origin, e.g. soy, almond, pea, potato, and wheat protein hydrolysates, are obtainable, for example, under the trademarks Gluadin® (Cognis), DiaMin® (Diamalt), Lexein® (Inolex), Hydrosoy® (Croda), Hydrolupin® (Croda), Hydrosesame® (Croda), Hydrotritium® (Croda), and Crotein® (Croda).

Although the use of protein hydrolysates per se is preferred, it is also optionally possible to use instead of them, if applicable, amino-acid mixtures obtained in different fashion. It is likewise possible to use derivatives of protein hydrolysates, for example in the form of their fatty acid condensation products. Such products are marketed, for example, under the designations Lamepon® (Cognis), Lexein® (Inolex), Crolastin® (Croda), Crosilk® (Croda), or Crotein® (Croda).

The teaching according to the present invention of course encompasses all isomeric forms, such as cis-trans isomers, diastereomers, and chiral isomers.

It is also possible according to the present invention to utilize a mixture of several protein hydrolysates.

The protein hydrolysates are used, for example, in concentrations from 0.01 wt % to 20 wt %, by preference from 0.05 wt % to 15 wt %, and very particularly preferably in quantities from 0.05 wt % to 5 wt %, based in each case on the entire application preparation.

Silicone oils and/or silicone gums are also suitable care-providing substances.

Silicone oils or silicone gums that are suitable according to the present invention are, in particular, dialkyl- and alkylaryisiloxanes such as, for example, dimethylpolysiloxane and methylphenylpolysiloxane, as well as alkoxylated, quaternized, or even anionic derivatives thereof. Cyclic and linear polydialkylsiloxanes, alkoxylated and/or aminated derivatives thereof, dihydroxypolydimethylsiloxanes, and polyphenylalkylsiloxanes are preferred.

Silicone oils produce a very wide variety of effects. For example, they simultaneously influence dry and wet combability, the feel of the dry and wet hair, and shine. The skilled artisan understands the term “silicone oils” to mean several structures of organosilicon compounds. It is understood firstly to mean the dimethiconols (S1). These can be both linear and branched, and also cyclic or cyclic and branched. Linear dimethiconois can be represented by the following structural formula (S1-I):

(HOSiR¹₂)—O—(SiR₂²—O—)ₓ—(SiR¹₂OH) (S1-I)

Branched dimethiconols can be represented by the structural formula (S1-II):

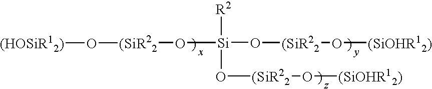

The R¹ and R² radicals each denote, mutually independently, hydrogen, a methyl radical, a C₂ to C₃₀ linear, saturated or unsaturated hydrocarbon radical, a phenyl radical, and/or an aryl radical. Non-limiting examples of the radicals represented by R¹ and R² include alkyl radicals such as methyl, ethyl, propyl, isopropyl, butyl, isobutyl, pentyl, isopentyl, neopentyl, amyl, isoamyl, hexyl, isohexyl and the like; alkenyl radicals such as vinyl, halovinyl, alkylvinyl, allyl, haloallyl, alkylallyl; cycloalkyl radicals such as cyclobutyl, cyclopentyl, cyclohexyl, and the like; phenyl radicals, benzyl radicals, halogenated hydrocarbon radicals such as 3-chloropropyl, 4-bromobutyl, 3,3,3-trifluoropropyl, chlorocyclohexyl, bromophenyl, chlorophenyl, and the like, and sulfur-containing radicals such as mercaptoethyl, mercaptopropyl, mercaptohexyl, mercaptophenyl, and the like; by preference, R¹ and R² are an alkyl radical that contains 1 to approximately 6 carbon atoms, and particularly preferably R¹ and R² are methyl. The numbers x, y, and z are integers and range, mutually independently in each case, from 0 to 50,000. The molecular weights of the dimethiconols are between 1000 D and 10,000,000 D. The viscosities are between 100 and 10,000,000 cPs, measured at 25° C. using a glass capillary viscosimeter in accordance with Dow Corning Corporate Test Method CTM 0004 of Jul. 20, 1970. Preferred viscosities are between 1000 and 5,000,000 cPs; very particularly preferred viscosities are between 10,000 und 3,000,000 cPs. The most preferred range is between 50,000 und 2,000,000 cPs.

The following commercial products are recited as examples of such products. Botanisil NU-150M (Botanigenics), Dow Corning 1-1254 Fluid, Dow Corning 2-9023 Fluid, Dow Corning 2-9026 Fluid, Ultrapure Dimethiconol (Ultra Chemical), Unisil SF-R (Universal Preserve), X-21-5619 (Shin-Etsu Chemical Co.), Abil OSW 5 (Degussa Care Specialties), ACC DL-9430 Emulsion (Taylor Chemical Company), AEC Dimethiconol & Sodium Dodecylbenzenesulfonate (A & E Connock (Perfumery & Cosmetics) Ltd.), B C Dimethiconol Emulsion 95 (Basildon Chemical Company, Ltd.), Cosmetic Fluid 1401, Cosmetic Fluid 1403, Cosmetic Fluid 1501, Cosmetic Fluid 1401DC (all the aforesaid Chemsil Silicones, Inc.), Dow Corning 1401 Fluid, Dow Corning 1403 Fluid, Dow Corning 1501 Fluid, Dow Corning 1784 HVF Emulsion, Dow Corning 9546 Silicone Elastomer Blend (all the aforesaid Dow Corning Corporation), Dub Gel SI 1400 (Stearinerie Dubois Fils), HVM 4852 Emulsion (Crompton Corporation), Jeesilc 6056 (Jeen international Corporation), Lubrasil, Lubrasil DS (both Guardian Laboratories), Nonychosine E, Nonychosine V (both Exsymol), SanSurf Petrolatum-25, Satin Finish (both Collaborative Laboratories, Inc.), Silatex-D30 (Cosmetic Ingredient Resources), Silsoft 148, Silsoft E-50, Silsoft E-623 (all the aforesaid Crompton Corporation), SM555, SM2725, 5M2765, SM2785 (all the aforesaid GE Silicones), Taylor T-Sil CD-1, Taylor TME-4050E (all Taylor Chemical Company), TH V 148 (Crompton Corporation), Tixogel CYD-1429 (Sud-Chemie Performance Additives), Wacker-Belsil CM 1000, Wacker-Belsil CM 3092, Wacker-Belsil CM 5040, Wacker-Belsil DM 3096, Wacker-Belsil DM 3112 VP, Wacker-Belsil DM 8005 VP, Wacker-Belsil DM 60081 VP (all the aforesaid Wacker-Chemie GmbH).

Dimethicones (S2) constitute the second group of silicones that can be contained according to the present invention. They can be both linear and branched, and also cyclic or cyclic and branched. Linear dimethicones can be represented by the following structural formula (S2-I):

(SiR¹₃)—O—(SiR¹R²—O—)ₓ—(SiR¹₃) (S2-I)

Branched dimethicones can be represented by the structural formula (S2-II):

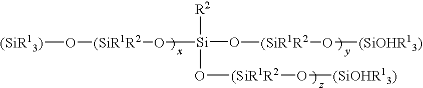

The R¹ and R² radicals each denote, mutually independently, hydrogen, a methyl radical, a C₂ to C₃₀ linear, saturated or unsaturated hydrocarbon radical, a phenyl radical, and/or an aryl radical. Non-limiting examples of the radicals represented by R¹ and R² include alkyl radicals such as methyl, ethyl, propyl, isopropyl, butyl, isobutyl, pentyl, isopentyl, neopentyl, amyl, isoamyl, hexyl, isohexyl and the like; alkenyl radicals such as vinyl, halovinyl, alkylvinyl, allyl, haloallyl, alkylallyl; cycloalkyl radicals such as cyclobutyl, cyclopentyl, cyclohexyl, and the like; phenyl radicals, benzyl radicals, halogenated hydrocarbon radicals such as 3-chloropropyl, 4-bromobutyl, 3,3,3-trifluoropropyl, chlorocyclohexyl, bromophenyl, chlorophenyl, and the like, and sulfur-containing radicals such as mercaptoethyl, mercaptopropyl, mercaptohexyl, mercaptophenyl, and the like; by preference, R¹ and R² are an alkyl radical that contains 1 to approximately 6 carbon atoms, and particularly preferably R¹ and R² are methyl. The numbers x, y, and z are integers and range, mutually independently in each case, from 0 to 50,000. The molecular weights of the dimethicones are between 1000 D and 10,000,000 D. The viscosities are between 100 and 10,000,000 cPs, measured at 25° C. using a glass capillary viscosimeter in accordance with Dow Corning Corporate Test Method CTM 0004 of Jul. 20, 1970. Preferred viscosities are between 1000 and 5,000,000 cPs; particularly preferred viscosities are between 10,000 und 3,000,000 cPs. Very particularly preferably, the viscosity is in the range between 50,000 und 2,000,000 cPs.

Dimethicone copolyols (S3) constitute a further group of silicones that are suitable. Dimethicone copolyols can be represented by the following structural formulas:

(SiR¹₃)—O—(SiR²₂—O—)ₓ(SiR²PE-O—)_{y}—(SiR¹₃) (S3-I),

PE-(SiR¹₂)—O—(SiR²₂)ₓ—(SiR¹₂)—PE (S3-II)

Branched dimethicone copolyols can be represented by the structural formula (S3-III):

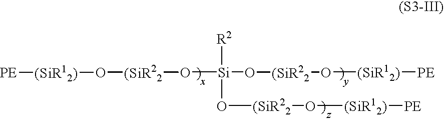

or by the structural formula (S3-IV):

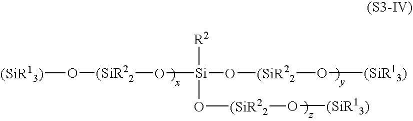

The R¹ and R² radicals each denote, mutually independently, hydrogen, a methyl radical, a C₂ to C₃₀ linear, saturated or unsaturated hydrocarbon radical, a phenyl radical, and/or an aryl radical. Non-limiting examples of the radicals represented by R¹ and R² include alkyl radicals such as methyl, ethyl, propyl, isopropyl, butyl, isobutyl, pentyl, isopentyl, neopentyl, amyl, isoamyl, hexyl, isohexyl and the like; alkenyl radicals such as vinyl, halovinyl, alkylvinyl, allyl, haloallyl, alkylallyl; cycloalkyl radicals such as cyclobutyl, cyclopentyl, cyclohexyl, and the like; phenyl radicals, benzyl radicals, halogenated hydrocarbon radicals such as 3-chloropropyl, 4-bromobutyl, 3,3,3-trifluoropropyl, chlorocyclohexyl, bromophenyl, chlorophenyl, and the like, and sulfur-containing radicals such as mercaptoethyl, mercaptopropyl, mercaptohexyl, mercaptophenyl, and the like; by preference, R¹ and R² are an alkyl radical that contains 1 to approximately 6 carbon atoms, and particularly preferably R¹ and R² are methyl. PE denotes a polyoxyalkylene radical. Preferred polyoxyalkylene radicals are derived from ethylene oxide, propylene oxide, and glycerol. The numbers x, y, and z are integers and range, mutually independently in each case, from 0 to 50,000. The molecular weights of the dimethicones are between 1000 D and 10,000,000 D. The viscosities are between 100 and 10,000,000 cPs, measured at 25° C. using a glass capillary viscosimeter in accordance with Dow Corning Corporate Test Method CTM 0004 of Jul. 20, 1970. Preferred viscosities are between 1000 and 5,000,000 cPs; very particularly preferred viscosities are between 10,000 und 3,000,000 cPs. The most preferred range is between 50,000 und 2,000,000 cPs.

Corresponding dimethicone copolyols are commercially obtainable and are marketed, for example, by the Dow Corning company under the designation Dow Corning® 5330 Fluid.

The dimethiconols, dimethicones, and/or dimethicone copolymers can, of course, already be present as an emulsion. The corresponding emulsion of the dimethiconols, dimethicones, and/or dimethicone copolyols can be manufactured both after manufacture of the corresponding dimethiconols, dimethicones, and/or dimethicone copolyols, from them and using usual emulsification methods known to the skilled artisan. For this purpose both cationic, anionic, nonionic, or zwitterionic surfactants and emulsifiers can be used, as auxiliaries, as adjuvants for manufacture of the corresponding emulsions. The emulsions of the dimethiconols, dimethicones, and/or dimethicone copolyols can of course also be manufactured directly by way of an emulsion polymerization method. Such methods are also very familiar to the skilled artisan.

If the dimethiconols, dimethicones, and/or dimethicone copolyols are used as an emulsion, the droplet size of the emulsified particles is then, according to the present invention, 0.01 to 10,000 μm, preferably 0.01 to 100 μm, particularly preferably 0.01 to 20 μm, and very particularly preferably 0.01 to 10 μm. The particle size is determined using the light-scattering method.

If branched dimethiconols, dimethicones, and/or dimethicone copolyols are used, this is to be understood to mean that the branching is greater than a random branching that occurs randomly as a result of contaminants in the respective monomers. “Branched” dimethiconols, dimethicones, and/or dimethicone copolyols are therefore to be understood, for purposes of the present invention, to mean that the degree of branching is greater than 0.01%. A degree of branching greater than 0.1% is preferred, and very particularly preferably it is greater than 0.5%. The degree of branching is determined from the ratio of unbranched monomers to the branching monomers, i.e. the quantity of tri- and tetrafunctional siloxanes. Both low-branching and high-branching dimethiconols, dimethicones, and/or dimethicone copolyols can be very particularly preferred according to the present invention.

Suitable silicones are, in addition, aminofunctional silicones (S4), in particular the silicones that are grouped under the INCI name Amodimethicone. These are to be understood as silicones that comprise at least one, optionally substituted, amino group.

Such silicones can be described, for example, by the formula (S4-I)

M(RₐQ_{b}SiO_{(4-a-b)/2)x}(R_{c}SiO_{(4-c)/2)y}M (S4-I);

in the above formula, R is a hydrocarbon or hydrocarbon radical having 1 to approximately 6 carbon atoms, Q is a polar radical of the general formula —R¹Z, in which R¹ is a bivalent bonding group that is bound to hydrogen and to the Z radical, assembled from carbon and hydrogen atoms, carbon, hydrogen, and oxygen atoms, or carbon, hydrogen, and nitrogen atoms, and Z is an organic aminofunctional radical that contains at least one aminofunctional group; “a” assumes values in the range from approximately 0 to approximately 2, “b” assumes values in the range from approximately 1 to approximately 3, “a”+“b” is less than or equal to 3, and “c” is a number in the range from approximately 1 to approximately 3, and x is a number in the range from 1 to approximately 2,000, preferably from approximately 3 to approximately 50, and most preferably from approximately 3 to approximately 25, and y is a number in the range from approximately 20 to approximately 10,000, preferably from approximately 125 to approximately 10,000, and most preferably from approximately 150 to approximately 1,000, and M is a suitable silicone terminal group that is known in the existing art, by preference trimethylsiloxy. Non-limiting examples of the radicals represented by R include alkyl radicals such as methyl, ethyl, propyl, isopropyl, butyl, isobutyl, amyl, isoamyl, hexyl, isohexyl and the like; alkenyl radicals such as vinyl, halovinyl, alkylvinyl, allyl, haloallyl, alkylallyl; cycloalkyl radicals such as cyclobutyl, cyclopentyl, cyclohexyl and the like; phenyl radicals, benzyl radicals, halocarbon radicals such as 3-chloropropyl, 4-bromobutyl, 3,3,3-trifluoropropyl, chlorocyclohexyl, bromophenyl, chlorophenyl, and the like, and sulfur-containing radicals such as mercaptoethyl, mercaptopropyl, mercaptohexyl, mercaptophenyl and the like; R is preferably an alkyl radical that contains 1 to approximately 6 carbon atoms, and R is most preferably methyl. Examples of R¹ include methylene, ethylene, propylene, hexamethylene, decamethylene, —CH₂CH(CH₃)CH₂—, phenylene, naphthylene, —CH₂CH₂SCH₂CH₂—, —CH₂CH₂OCH₂—, —OCH₂CH₂—, —OCH₂CH₂CH₂—, —CH₂CH(CH₃)C(O)OCH₂—, —(CH₂)₃C(O)OCH₂CH₂—, —C₆H₄C₆H₄—, —C₆H₄CH₂C₆H₄—, and —(CH₂)₃C(O)SCH₂CH₂—.

Z is an organic aminofunctional radical containing at least one functional amino group. One possible formula for Z is NH(CH₂)_{z}NH₂, in which z denotes an integer from 1 to 50. Another possible formula for Z is —NH(CH₂)_{z}NH(CH₂)_{zz}, in which both z and zz denote, mutually independently, an integer from 1 to 50; this structure encompasses diamino ring structures such as piperazinyl. Z is particularly preferably a —NHCH₂CH₂NH₂ radical. Another possible formula for Z is —N(CH₂)_{z}NX¹X² or —NX¹NX², in which X¹ and X² are selected, mutually independently in each case, from hydrogen and a hydrocarbon radical having from 1 to approximately 6 carbon atoms.

Very particularly preferably, Q denotes a polar aminofunctional radical of the formula —CH₂CH₂CH₂NHCH₂CH₂NH₂.

The molar ratio of the RₐQ_{b}SiO_{(4-a-b)/2} units to the R_{c}SiO_{(4-c)/2} units is in the range from approximately 1:2 to 1:65, preferably from approximately 1:5 to approximately 1:65, and most preferably from approximately 1:15 to approximately 1:20. If one or more silicones of the above formula are used, the different variable substituents in the above formula can then be different in the different silicone components that are present in the silicone mixture.

Preferred aminofunctional silicones correspond to formula (S4-II)

R′ₐG₃₋ₐ-Si(OSiG₂)ₙ-(OSiG_{b}R′_{2-b})ₘ—O—SiG3-a-R′ₐ (S4-II),

in which
G is —H, a phenyl group, —OH, —O—CH₃, —CH₃, —CH₂CH₃, —CH₂CH₂CH₃, —CH(CH₃)₂, —CH₂CH₂CH₂CH₃, —CH₂CH(CH₃)₂, —CH(CH₃)CH₂CH₃, —C(CH₃)₃;
a denotes a number between 0 and 3, in particular 0;
b denotes a number between 0 and 1, in particular 1,
m and n are numbers whose sum (m+n) is between 1 and 2000, preferably between 50 and 150, n preferably assuming values from 0 to 1999 and in particular from 49 to 149, and m preferably assuming values from 1 to 2000, in particular from 1 to 10;
R′ is a monovalent radical selected from

—N(R″)—CH₂—CH₂—N(R″)₂

—N(R″)₂

—N⁺(R″)₃A⁻

—N⁺H(R″)₂ A⁻

—N⁺H₂(R−)A⁻

—N(R″)—CH₂—CH₂—N⁺R″H₂A⁻,
each R″ denoting identical or different radicals from the group of —H, phenyl, benzyl, the C₁₋₂₀ alkyl radicals, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, and A⁻ representing an anion that is preferably selected from chloride, bromide, iodide, or methosulfate.

Particularly preferred aminofunctional silicones correspond to formula (S4-III)

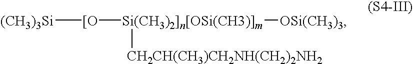

in which m and n are numbers whose sum (m+n) is between 1 and 2000, preferably between 50 and 150, n preferably assuming values from 0 to 1999 and in particular from 49 to 149, and m preferably assuming values from 1 to 2000, in particular from 1 to 10.

These silicones are referred to according to the INCI declaration as Trimethylsilylamodimethicone.

Also particularly preferred are aminofunctional silicones of formula (S4-IV)

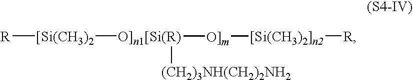

in which R denotes —OH, —O—CH₃, or a —CH₃ group, and m, n1, and n2 are numbers whose sum (m+n1+n2) is between 1 and 2000, preferably between 50 and 150, the sum (n1+n2) preferably assuming values from 0 to 1999 and in particular from 49 to 149, and m preferably assuming values from 1 to 2000, in particular from 1 to 10.

These silicones are referred to according to the INCI declaration as Amodimethicone and are available, for example, in the form of an emulsion as the commercial product Dow Corning® 949, mixed with a cationic and a nonionic surfactant.

Those aminofunctional silicones that have an amine number above 0.25 meq/g, preferably above 0.3 meq/g, and particularly preferably above 0.4 meq/g are used by preference. The amine number denotes the milliequivalent of amine per gram of the aminofunctional silicone; it can be ascertained by titration, and is also indicated with the “mg KOH/g” unit.

Further suitable silicones are, for example:
oligomeric polydimethylcyclosiloxanes (INCI name: Cyclomethicone), in particular the tetrameric and the pentameric compound, which are marketed by Dow Corning as commercial products DC 245 Fluid, DC 344 and DC 345, respectively;
hexamethyidisiloxane (INCI name: Hexamethyldisiloxane), e.g. the product marketed under the designation Abil® K 520;
polyphenylmethylsiloxanes (INCI name: Phenyl Trimethicone), e.g. the commercial product DC 556 Cosmetic Grade Fluid of Dow Corning;
esters and partial esters of the silicone-glycol copolymers such as those marketed, for example, by the Fanning company under the commercial designation Fancorsil® LIM (INCI name. Dimethicone Copolyol Meadowfoamate);
anionic silicone oils such as, for example, the product Dow Corning® 1784.

The use of at least two different silicone derivatives is also possible. A combination of a volatile and a non-volatile silicone is preferred. Those silicones that exhibit a volatility equal to or greater than the volatility of cyclic pentameric dimethylsiloxane are “volatile” for purposes of the invention. Such combinations are also available as commercial products (e.g. Dow Corning® 1401, Dow Corning® 1403, and Dow Corning® 1501, in each case mixtures of a cyclomethicone and a dimethiconol).

Preferred mixtures of different silicones are, for example, dimethicones and dimethiconols, linear dimethicones, and cyclic dimethiconols. A very particularly preferred mixture of silicones is made up of at least one cyclic dimethiconol and/or dimethicone, at least one further non-cyclic dimethicone and/or dimethiconol, and at least one aminofunctional silicone.

If different silicones are used as a mixture, the mixing ratio is largely variable. Preferably, however, all the silicones used for mixing are utilized at a ratio from 5:1 to 1:5 in the case of a binary mixture. A ratio from 3:1 to 1:3 is particularly preferred. Very particularly preferred mixtures contain all the silicones contained in the mixture very largely at a ratio of approximately 1:1, based in each case on the quantity used in wt %.

The silicones are used preferably in quantities from 1 to 25 wt %, particularly preferably from 5 to 20 wt %, and particularly preferably from 7 to 15 wt %, based in each case on the entire product.

Care-providing polymers are likewise suitable as a care-providing substance. Be it noted explicitly at this juncture that a number of the polymers recited below also exhibit film-forming and/or setting properties, and have therefore already been described above as film-forming and/or setting polymers.

A first group of care-providing polymers is the cationic polymers. “Cationic polymers” are to be understood as polymers that comprise in the main chain and/or side chain a group that can be “temporarily” or “permanently” cationic. According to the present invention, those polymers that possess a cationic group regardless of the pH of the agent are referred to as “permanently cationic.” These are, as a rule, polymers that contain a quaternary nitrogen atom, for example in the form of an ammonium group. Preferred cationic groups are quaternary ammonium groups. In particular, those polymers in which the quaternary ammonium group is bound via a C₁₋₄ hydrocarbon group to a main polymer chain made up of acrylic acid, methacrylic acid, or derivatives thereof, have proven to be particularly suitable.

Homopolymers of the general formula (G1-I),

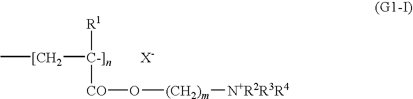

in which R¹═—H or —CH₃, R², R³ and R⁴ are selected, mutually independently, from C₁₋₄ alkyl, alkenyl, or hydroxyalkyl groups, m=1, 2, 3 or 4, n is a natural number, and X⁻ is a physiologically acceptable organic or inorganic anion, as well as copolymers made up substantially of the monomer units presented in formula (G1-I) as well as nonionogenic monomer units, are particularly preferred cationic polymers. In the context of these polymers, those for which at least one of the following conditions apply are preferred according to the present invention:
R¹ denotes a methyl group
R², R³ and R⁴ denote methyl groups
m has the value of 2.

Possibilities as physiologically acceptable counterions X⁻ are, for example, halide ions, sulfate ions, phosphate ions, methosulfate ions, and organic ions such as lactate, citrate, tartrate, and acetate ions. Halide ions, in particular chloride, are preferred.

A particularly suitable homopolymer is the poly(methacryloyloxyethyltrimethylammonium chloride) (crosslinked, if desired) having the INCI name Polyquaternium-37. The crosslinking can be accomplished, if desired, with the aid of polyolefinically unsaturated compounds, for example divinylbenzene, tetraallyloxyethane, methylene bisacrylamide, diallyl ether, polyallylpolyglyceryl ether, or allyl ethers of sugars or sugar derivatives such as erythritol, pentaerythritol, arabitol, mannitol, sorbitol, sucrose, or glucose. Methylene bisacrylamide is a preferred cross-linking agent.

The homopolymer is preferably used in the form of a nonaqueous polymer dispersion that should comprise a polymer proportion not less than 30 wt %. Such polymer dispersions are obtainable commercially under the designations Salcare® SC 95 (approx, 50% polymer proportion, further components: mineral oil (INCI name: Mineral Oil) and tridecylpolyoxypropylenepolyoxyethylene ether (INCI name: PPG-1-Trideceth-6)), and Salcare® SC 96 (approx. 50% polymer proportion, further components: mixture of diesters of propylene glycol with a mixture of caprylic and capric acid (INCI name: Propylene Glycol Dicaprylate/Dicaprate) and tridecylpolyoxypropylenepolyoxyethylene ether (INCI name: PPG-1-Trideceth-6)).

Copolymers having monomer units according to formula (G1-I) preferably contain acrylamide, methacrylamide, acrylic acid C₁₋₄ alkyl esters, and methacrylic acid C₁₋₄ alkyl esters as nonionogenic monomer units. Of these nonionogenic monomers, acrylamide is particularly preferred. These copolymers as well, as in the case of the homopolymers described above, can be crosslinked. A copolymer preferred according to the present invention is the crosslinked copolymer of acrylamide and methacryloyloxyethyltrimethylammonium chloride. Such copolymers, in which the monomers are present at a weight ratio of approximately 20:80, are commercially obtainable as an approx. 50% nonaqueous polymer dispersion under the designation Salcare® SC 92.

Additional preferred cationic polymers are, for example:
quaternized cellulose derivatives such as those obtainable commercially under the designations Celquat® and Polymer JR®. The compounds Celquat® H 100, Celquat® L 200, and Polymer JR® 400 are preferred quaternized cellulose derivatives;
cationic alkyl polyglycosides according to DE Patent 44 13 686;
cationized honey, for example the commercial product Honeyquat® 50;
cationic guar derivatives such as, in particular, the products marketed under the trade names Cosmedia® Guar and Jaguar®,
polysiloxanes having quaternary groups, such as, for example, the commercially obtainable products Q2-7224 (manufacturer: Dow Corning; a stabilized trimethylsilylamodimethicone), Dow Corning® 929 Emulsion (containing a hydroxylamino-modified silicone that is also referred to as Amodimethicone), SM-2059 (manufacturer: General Electric), SLM-55067 (manufacturer; Wacker), and Abil®-Quat 3270 and 3272 (manufacturer: Th. Goldschmidt; diquaternary polydimethylsiloxanes, Quaternium-80);
polymeric dimethyldiallylammonium salts and copolymers thereof with esters and amides of acrylic acid and methacrylic acid. The products available commercially under the designations Merquat® 100 (poly(dimethyldiallylammonium chloride)) and Merquat® 550 (dimethyldiallylammonium chloride/acrylamide copolymer) are examples of such cationic polymers;
copolymers of vinylpyrrolidone with quaternized derivatives of dialkylaminoalkyl acrylate and methacrylate, such as, for example, vinylpyrrolidone/dimethylaminoethyl methacrylate copolymers quaternized with diethyl sulfate. Such compounds are obtainable commercially under the designations Gafquat®734 and Gafquat®755;
vinylpyrrolidone/vinylimidazolium methochloride copolymers, such as those offered under the designations Luviquat® FC 370, FC 550, FC 905, and HM 552,
quaternized poly(vinylalcohol); and
the polymers known under the designations Polyquaternium-2, Polyquaternium-17, Polyquaternium-18, and Polyquaternium-27, having quaternary nitrogen atoms in the main polymer chain.

The polymers known under the designations Polyquaternium-24 (commercial product e.g. Quatrisoft® LM 200) can similarly be used as cationic polymers. Likewise usable according to the present invention are the copolymers of vinylpyrrolidone such as those available as the commercial products Copolymer 845 (manufacturer: ISP), Gaffix® VC 713 (manufacturer: ISP), Gafquat® ASCP 1011, Gafquat® HS 110, Luviquat® 8155, and Luviquat® MS 370.

Additional cationic polymers usable according to the present invention are the so-called “temporarily cationic” polymers. These polymers usually contain an amino group that is present at certain pH values as a quaternary ammonium group and therefore cationically. Chitosan and its derivatives, such as those readily available commercially, for example, under the commercial designations Hydagen® CMF, Hydagen® HCMF, Kytamer® PC, and Chitolam® NIB/101 are, for example, preferred.

Cationic polymers that are preferred for use according to the present invention are cationic cellulose derivatives and chitosan and its derivatives, in particular the commercial products Polymer® JR 400, Hydagen® HCMF, and Kytamer® PC, cationic guar derivatives, cationic honey derivatives, in particular the commercial product Honeyquat® 50, cationic alkyl polyglycosides, and polymers of the Polyquaternium-37 type.

Also to be listed among the cationic polymers are cationized protein hydrolysates, in which context the underlying protein hydrolysate can derive from animals, for example from collagen, milk, or keratin, from plants, for example from wheat, corn, rice, potatoes, soy, or almonds, from marine life forms, for example from fish collagen or algae, or from biotechnologically obtained protein hydrolysates. The protein hydrolysates serving as the basis for the cationic derivatives according to the present invention can be obtained from the corresponding proteins by way of a chemical, in particular alkaline or acid, hydrolysis, by an enzymatic hydrolysis, and/or by a combination of both types of hydrolysis. The hydrolysis of proteins results, as a rule, in a protein hydrolysate having a molecular weight distribution from approximately 100 dalton up to several thousand dalton. Those cationic protein hydrolysates whose underlying protein component has a molecular weight from 100 to 25,000 dalton, preferably 250 to 5,000 dalton, are preferred. Also to be understood as cationic protein hydrolysates are quaternized amino acids and mixtures thereof. Quaternization of the protein hydrolysates or amino acids is often carried out by means of quaternary ammonium salts such as, for example, N,N-dimethyl-N-(n-alkyl)-N-(2-hydroxy-3-chloro-n-propyl)ammonium halides. The cationic protein hydrolysates can furthermore also be further derivatized. Typical examples that may be mentioned of cationic protein hydrolysates and derivatives according to the present invention are the following products listed under the INCI names in the “International Cosmetic Ingredient Dictionary and Handbook” (seventh edition 1997, The Cosmetic, Toiletry, and Fragrance Association, 1101 17^{th} Street, N.W., Suite 300, Washington, DC 20036-4702) and available commercially: Cocodimonium Hydroxypropyl Hydrolyzed Collagen, Cocodimonium Hydroxypropyl Hydrolyzed Casein, Cocodimonium Hydroxypropyl Hydrolyzed Collagen, Cocodimonium Hydroxypropyl Hydrolyzed Hair Keratin, Cocodimonium Hydroxypropyl Hydrolyzed Keratin, Cocodimonium Hydroxypropyl Hydrolyzed Rice Protein, Cocodimonium Hydroxypropyl Hydrolyzed Soy Protein, Cocodimonium Hydroxypropyl Hydrolyzed Wheat Protein, Hydroxypropyl Arginine Lauryl/Myristyl Ether HCl, Hydroxypropyltrimonium Gelatin, Hydroxypropyltrimonium Hydrolyzed Casein, Hydroxypropyltrimonium Hydrolyzed Collagen, Hydroxypropyltrimonium Hydrolyzed Conchiolin Protein, Hydroxypropyltrimonium Hydrolyzed Keratin, Hydroxypropyltrimonium Hydrolyzed Rice Bran Protein, Hydroxypropyltrimonium Hydrolyzed Soy Protein, Hydroxypropyl Hydrolyzed Vegetable Protein, Hydroxypropyltrimonium Hydrolyzed Wheat Protein, Hydroxypropyltrimonium Hydrolyzed Wheat Protein/Sioxysilicate, Laurdimonium Hydroxypropyl Hydrolyzed Soy Protein, Laurdimonium Hydroxypropyl Hydrolyzed Wheat Protein, Laurdimonium Hydroxypropyl Hydrolyzed Wheat Protein/Siloxysilicate, Lauryldimonium Hydroxypropyl Hydrolyzed Casein, Lauryldimonium Hydroxypropyl Hydrolyzed Collagen, Lauryldimonium Hydroxypropyl Hydrolyzed Keratin, Lauryldimonium Hydroxypropyl Hydrolyzed Soy Protein, Steardimonium Hydroxypropyl Hydrolyzed Casein, Steardimonium Hydroxypropyl Hydrolyzed Collagen, Steardimonium Hydroxypropyl Hydrolyzed Keratin, Steardimonium Hydroxypropyl Hydrolyzed Rice Protein, Steardimonium Hydroxypropyl Hydrolyzed Soy Protein, Steardimonium Hydroxypropyl Hydrolyzed Vegetable Protein, Steardimonium Hydroxypropyl Hydrolyzed Wheat Protein, Steartrimonium Hydroxyethyl Hydrolyzed Collagen, Quaternium-76 Hydrolyzed Collagen, Quaternium-79 Hydrolyzed Collagen, Quaternium-79 Hydrolyzed Keratin, Quaternium-79 Hydrolyzed Milk Protein, Quaternium-79 Hydrolyzed Soy Protein, Quaternium-79 Hydrolyzed Wheat Protein.

The plant-based cationic protein hydrolysates and derivatives are very particularly preferred.

Amphoteric polymers used in preferred fashion are those polymerizates made up substantially of
(a) Monomers having quaternary ammonium groups of the general formula (II)

R¹—CH═CR²—CO-Z-(CₙH₂ₙ)—N⁽⁺⁾R³R⁴R⁵ A⁽⁻⁾ (II)

in which R¹ and R², mutually independently, denote hydrogen or a methyl group, and R³, R⁴ and R⁵, each mutually independently, denote an alkyl group having 1 to 4 carbon atoms, Z denotes an NH group or an oxygen atom, n is an integer from 2 to 5, and A⁽⁻⁾ is the anion of an organic or inorganic acid; and
(b) monomeric carboxylic acids of the general formula (III)

R⁶—CH═CR⁷—COOH (III)

in which R⁶ and R⁷, mutually independently, denote hydrogen or a methyl group.

These compounds can be used according to the present invention both directly and in the form of salts that are obtained by neutralization of the polymerizates, for example using an alkaline hydroxide. Regarding the details of manufacture of these polymerizates, reference is expressly made to the content of DE Unexamined Application 39 29 973. Those polymerizates in which monomers of type (a) are used in which R³, R⁴, and R⁵ are methyl groups, Z is an NH group, and A⁽⁻⁾ is a halide, methoxysulfate, or ethoxysulfate ion, are very particularly preferred; acrylamidopropyltrimethylammonium chloride is a particularly preferred monomer (a). Acrylic acid is preferably used as monomer (b) for the aforesaid polymerizates.

The care-providing cationic polymers are used preferably in a quantity from 0.01 to 5 wt %, in particular in a quantity from 0.1 to 2 wt %, based in each case on the entire application preparation.

The product according to the present invention can furthermore contain as a care-providing substance at least one vitamin, provitamin, vitamin precursor, and/or one of their derivatives.

Those vitamins, provitamins, and vitamin precursors that are usually assigned to groups A, B, C, E, F, and H are preferred according to the present invention.

The group of substances referred to as “vitamin A” includes retinol (vitamin A₁) as well as 3,4-didehydroretinol (vitamin A₂). β-Carotene is the provitamin of retinal. Vitamin A components that are suitable according to the present invention are, for example, vitamin A acid and its esters, vitamin A aldehyde, and vitamin A alcohol as well as esters thereof such as the palmitate and acetate. The products contain the vitamin A component preferably in quantities from 0.05 to 1 wt % based on the entire application preparation.

Members of the vitamin B group or vitamin B complex are, among others:
Vitamin B₁ (thiamine)
Vitamin B₂ (riboflavin)
Vitamin B₃. The compounds nicotinic acid and nicotinic acid amide (niacinamide) are often listed under this designation. Nicotinic acid amide is preferred according to the present invention; it is contained in the agents according to the present invention preferably in quantities from 0.05 to 1 wt % based on the entire application preparation.
Vitamin B₅ (pantothenic acid, panthenol, and pantolactone). In the context of this group, panthenol and/or pantolactone are preferably used. Derivatives of panthenol usable according to the present invention are, in particular, the esters and ethers of panthenol as well as cationically derivatized panthenols. Individual representatives are, for example, panthenol triacetate, panthenol monoethyl ether and its monoacetate, and the cationic panthenol derivatives disclosed in WO 92/13829. The aforesaid compounds of the vitamin B₅ type are contained in the products according to the present invention preferably in quantities from 0.05 to 10 wt % based on the entire application preparation. Quantities from 0.1 to 5 wt % are particularly preferred.
Vitamin B₆ (pyridoxine as well as pyridoxamine and pyridoxal). The aforesaid compounds of the vitamin B₆ type are contained in the products according to the present invention preferably in quantities from 0.01 to 5 wt % based on the entire application preparation Quantities from 0.05 to 1 wt % are particularly preferred.

Vitamin C (ascorbic acid). Vitamin C is utilized in the products used according to the present invention preferably in quantities from 0.1 to 3 wt % based on the entire application preparation. Utilization in the form of the palmitic acid ester, the glucosides or the phosphates may be preferred. Utilization in combination with tocopherols can likewise be preferred.

Vitamin E (tocopherols, in particular α-tocopherol). Tocopherol and its derivatives, which include in particular the esters such as the acetate, nicotinate, phosphate, and succinate, are contained in the products according to the present invention preferably in quantities from 0.05 to 1 wt % based on the entire application preparation.

Vitamin F. The term “vitamin F” is usually understood as essential fatty acids, in particular linoleic acid, linolenic acid, and arachidonic acid.

Vitamin H. This is the term used for (3aS,4S,6aR)-2-oxohexahydrothieno[3,4-d]-imidazole-4-valeric acid, for which the trivial name “biotin” has nevertheless since become established. Biotin is contained in the products according to the present invention preferably in quantities from 0.0001 to 1.0 wt %, in particular in quantities from 0.001 to 0.01 wt %, based in each case on the entire application preparation.

The products according to the present invention preferably contain vitamins, provitamins, and vitamin precursors from groups A, B, C, E and H.

Panthenol, pantolactone, pyridoxine and its derivatives, as well as nicotinic acid amide and biotin, are particularly preferred.

D-panthenol is used very particularly preferably as a care-providing substance, if applicable in combination with at least one of the aforesaid silicone derivatives.

The products according to the present invention can furthermore contain at least one plant extract as a care-providing substance.

These extracts are usually produced by extraction of the entire plant. In individual cases, however, it may also be preferred to produce the extracts exclusively from blossoms and/or leaves of the plant.

With regard to the plant extracts usable according to the present invention, reference is made in particular to the extracts that are listed in the table beginning on page 44 of the 3rd edition of the Guideline for declaring the contents of cosmetic agents [Leitfaden zur Inhaltsstoffdeklaration kosmetischer Mittel], published by the Association of the personal hygiene and washing agents industry [Industrieverband Körperpflege- und Waschmittel e.V. (IKW)], Frankfurt.

According to the present invention the extracts from green tea, oak bark, nettle, hamamelis, hops, henna, chamomile, burdock root, horsetail, hawthorn, linden blossoms, almond, aloe vera, pine needles, horse chestnut, sandalwood, juniper, coconut, mango, apricot, lemon, wheat, kiwi fruit, melon, orange, grapefruit, salvia, rosemary, birch, mallow, lady's-smock, wild thyme, yarrow, thyme, lemon balm, restharrow, coltsfoot, hibiscus, meristem, ginseng, and ginger root are especially preferred.

Particularly preferred are the extracts from green tea, oak bark, nettle, hamamelis, hops, chamomile, burdock root, horsetail, linden blossoms, almond, aloe vera, coconut, mango, apricot, lemon, wheat, kiwi fruit, melon, orange, grapefruit, salvia, rosemary, birch, lady's-smock, wild thyme, yarrow, restharrow, meristem, ginseng, and ginger root.

The extracts from green tea, almond, aloe vera, coconut, mango, apricot, lemon, wheat, kiwi fruit, and melon are very particularly suitable.

Water, alcohols, and mixtures thereof can be used as extraction agents for manufacturing the aforesaid plant extracts. Among the alcohols, lower alcohols such as ethanol and isopropanol, but in particular polyvalent alcohols such as ethylene glycol and propylene glycol, both as the only extraction agent and mixed with water, are preferred. Plant extracts based on water/propylene glycol at a ratio from 1:10 to 10:1 have proven particularly suitable.

According to the present invention the plant extracts can be used in both pure and diluted form, If they are used in diluted form, they usually contain approx. 2 to 80 wt % active substance, and contain as a solvent the extraction agent or extraction agent mixture used to obtain them.

It may furthermore be preferred to use mixtures of several, in particular two, different plant extracts.

A number of carboxylic acids are also suitable as a care-providing substance.

Short-chain carboxylic acids can be particularly advantageous for purposes of the invention. “Short-chain” carboxylic acids and derivatives thereof are understood, for purposes of the invention, as carboxylic acids that can be saturated or unsaturated and/or straight-chain or branched or cyclic and/or aromatic and/or heterocyclic, and have a molecular weight below 750. Saturated or unsaturated straight-chain or branched carboxylic acids having a chain length of 1 to 16 carbon atoms in the chain may be preferred for purposes of the invention; those having a chain length of 1 to 12 carbon atoms in the chain are very particularly preferred.

The short-chain carboxylic acids for purposes of the invention can comprise one, two, three, or more carboxy groups. Carboxylic acids having multiple carboxy groups, in particular di- and tricarboxylic acids, are preferred for purposes of the invention. The carboxy groups can be present entirely or partly as an ester, acid anhydride, lactone, amide, imidic acid, lactam, lactim, dicarboximide, carbohydrazide, hydrazone, hydroxam, hydroxime, amidine, amide oxime, nitrile, or phosphonic or phosphate ester. The carboxylic acids usable according to the present invention can of course be substituted along the carbon chain or the ring structure. Among the substituents of the carboxylic acids usable according to the present invention may be listed, for example, C₁ to C₈ alkyl, C₂ to C₈ alkenyl, aryl, aralkyl and aralkenyl, hydroxymethyl, C₂ to C₈ hydroxyalkyl, C₂ to C₈ hydroxyalkenyl, aminomethyl, C₂ to C₈ aminoalkyl, cyano, formyl, oxo, thioxo, hydroxy, mercapto, amino, carboxy or imino groups. Preferred substituents are C₁ to C₈ alkyl, hydroxymethyl, hydroxy, amino and carboxy groups. Substituents in the α-position are particularly preferred. Very particularly preferred substituents are hydroxy, alkoxy, and amino groups, in which context the amino function can be further substituted, if applicable, with alkyl, aryl, aralkyl, and/or alkenyl radicals. Furthermore, the phosphonic and phosphate esters are likewise preferred carboxylic acid derivatives.

The following may be mentioned as examples of carboxylic acids usable according to the present invention: formic acid, acetic acid, propionic acid, butyric acid, isobutyric acid, valeric acid, isovaleric acid, pivalic acid, oxalic acid, malonic acid, succinic acid, glutaric acid, glyceric acid, glyoxylic acid, adipic acid, pimelic acid, suberic acid, azelaic acid, sebacic acid, propiolic acid, crotonic acid, isocrotonic acid, elaidic acid, maleic acid, fumaric acid, muconic acid, citraconic acid, mesaconic acid, camphoric acid, benzoic acid, o,m,p-phthalic acid, naphthoic acid, toluic acid, hydratropic acid, atropic acid, cinnamic acid, isonicotinic acid, nicotinic acid, bicarbamic acid, 4,4′-dicyano-6,6′-binicotinic acid, 8-carbamoyloctanoic acid, 1,2,4-pentanetricarboxylic acid, 2-pyrrolecarboxylic acid, 1,2,4,6,7-napthalenepentaacetic acid, malonaldehydic acid, 4-hydroxyphthalamidic acid, 1-pyrazolecarboxylic acid, gallic acid, or propanetricarboxylic acid, a dicarboxylic acid selected from the group formed by compounds of the general formula (N-I):

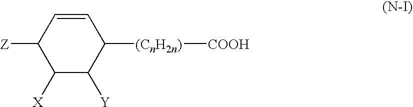

in which Z denotes a linear or branched alkyl or alkenyl group having 4 to 12 carbon atoms, n a number from 4 to 12, and one of the two groups X and Y denotes a COOH group and the other hydrogen or a methyl or ethyl radical, dicarboxylic acids of the general formula (N-I) that additionally bear 1 to 3 methyl or ethyl substituents on the cyclohexene ring, as well as dicarboxylic acids resulting from the dicarboxylic acids according to formula (N-I), in formal terms, by the attachment of one molecule of water to the double bond in the cyclohexene ring.

Dicarboxylic acids of formula (N-I) are known in the literature. A manufacturing method may be inferred, for example, from U.S. Pat. No. 3,753,968.

The dicarboxylic acids of formula (N-I) can be produced, for example, by reacting polyunsaturated dicarboxylic acids with unsaturated monocarboxylic acids in the form of a Diels-Alder cyclization. It is usual to proceed from a polyunsaturated fatty acid as a dicarboxylic acid component. Linoleic acid, accessible from natural fats and oils, is preferred. Acrylic acid in particular, but also e.g. methacrylic acid und crotonic acid, are preferred as a monocarboxylic acid component. Diels-Alder reactions usually result in isomer mixtures in which one component is present in excess. Both these isomer mixtures, as well as the pure compounds, can be used according to the present invention.

Also usable, in addition to the preferred dicarboxylic acids according to formula (N-I), are those dicarboxylic acids that differ from the compounds according to formula (N-I) by having 1 to 3 methyl or ethyl substituents on the cyclohexyl ring, or are formed from those compounds in formal terms by the attachment of one molecule of water to the double bond of the cyclohexene ring.

The dicarboxylic acid (mixture) resulting from the reaction of linoleic acid with acrylic acid has proven to be particularly effective according to the present invention. This is a mixture of 5- and 6-carboxy-4-hexyl-2-cyclohexene-1-octanoic acids. Such compounds are commercially obtainable under the designations Westvaco Diacid® 1550 and Westvaco Diacid® 1595 (manufacturer: Westvaco).

In addition to the short-chain carboxylic acids themselves that are listed above by way of example, physiologically acceptable salts thereof can also be used according to the present invention. Examples of such salts are the alkali, alkaline-earth, and zinc salts, as well as ammonium salts, among which the mono-, di-, and trimethyl-, -ethyl-, and hydroxyethylammonium salts are also to be understood in the context of the present Application. Very particularly preferably, however, acids neutralized with alkaline-reacting amino acids, for example arginine, lysine, ornithine, and histidine, can be used in the context of the invention. For formulation reasons, it may furthermore be preferred to select the carboxylic acid from the water-soluble representatives, in particular the water-soluble salts.

It is furthermore preferred according to the present invention to utilize 2-pyrrolidinone-5-carboxylic acid and its derivatives as a carboxylic acid. Particularly preferred are the sodium, potassium, calcium, magnesium or ammonium salts, in which context the ammonium ion carries, in addition to hydrogen, one to three C₁ to C₄ alkyl groups. The sodium salt is very particularly preferred. The quantities used in the agents according to the present invention are by preference 0.05 to 10 wt % based on the entire application preparation, particularly preferably 0.1 to 5 wt %, and in particular 0.1 to 3 wt %.

It is further preferred according to the present invention to use hydroxycarboxylic acids, and in this context in turn especially the dihydroxy-, trihydroxy- and polyhydroxycarboxylic acids, as well as the dihydroxy-, trihydroxy- and polyhydroxydi, tri- and -polycarboxylic acids. It has been found in this context that in addition to the hydroxycarboxylic acids, the hydroxycarboxylic acid esters, as well as mixtures of hydroxycarboxylic acids and their esters, and also polymeric hydroxycarboxylic acids and their esters, can be very particularly preferred. Preferred hydroxycarboxylic acid esters are, for example, full esters of glycolic acid, lactic acid, malic acid, tartaric acid, or citric acid. Additional hydroxycarboxylic acid esters that are suitable in principle are esters of β-hydroxypropionic acid, of tartronic acid, of D-gluconic acid, of saccharic acid, of muric acid, or of glucuronic acid. Suitable as alcohol components of these esters are primary, linear or branched aliphatic alcohols having 8 to 22 carbon atoms, i.e. for example fatty alcohols or synthetic fatty alcohols. The esters of C₁₂ to C₁₅ fatty alcohols are particularly preferred in this context. Esters of this type are obtainable commercially, e.g. under the trademark Cosmacol® of EniChem, Augusta Industriale. Particularly preferred polyhydroxypolycarboxylic acids are polylactic acid und polytartaric acid as well as esters thereof.

Ectoin or ectoin derivatives, allantoin, taurine, and/or bisabolol are also suitable as a care-providing substance.

The term “ectoin and ectoin derivatives” is understood, according to the present invention, as compounds of formula (IV):

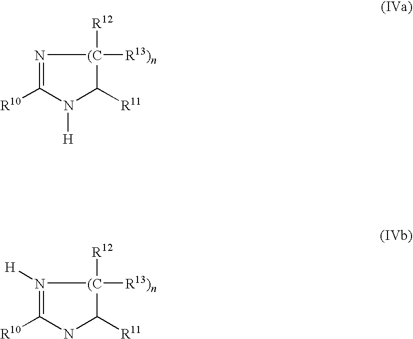

and/or physiologically acceptable salts thereof and/or an isomeric or stereoisomeric form, in which
R¹⁰ denotes a hydrogen atom, a branched or unbranched C₁ to C₄ alkyl radical, or a C₂ to C₄ hydroxyalkyl radical;
R¹¹ denotes a hydrogen atom, a —COOR¹⁴ grouping, or a —CO(NH)R¹⁴ grouping, in which context R¹⁴ can denote a C₁ to C₄ alkyl radical, an amino acid radical, or a dipeptide or tripeptide radical;
R¹² and R¹³ denote, mutually independently, a hydrogen atom, a C₁ to C₄ alkyl radical, or a hydroxy group, with the stipulation that the two radicals must not simultaneously denote a hydroxy group; and
n denotes an integer from 1 to 3.

Suitable physiologically acceptable salts of the general compounds according to formula (IVa) or (IVb) are, for example, the alkaline, alkaline-earth, ammonium, triethylamine, or tris-(2-hydroxyethyl)amine salts, as well as those that result from the reaction of compounds according to formula (IVa) or (IVb) with inorganic and organic acids such as hydrochloric acid, phosphoric acid, sulfuric acid, branched or unbranched, substituted or unsubstituted (for example with one or more hydroxy groups) C₁ to C₄ mono- or dicarboxylic acids, aromatic carboxylic acids and sulfonic acids such as acetic acid, citric acid, benzoic acid, maleic acid, fumaric acid, tartaric acid, and p-toluenesulfonic acid. Examples of particularly preferred physiologically acceptable salts are the Na, K, Mg, Ca, and ammonium salts of the compounds according to formula (IVa) or (IVb), as well as the salts that result from the reaction of compounds according to formula (IVa) or (IVb) with hydrochloric acid, acetic acid, citric acid, and benzoic acid.

Isomeric or stereoisomeric forms of the compounds according to formula (IVa) or (IVb) are understood, according to the present invention, as all optical isomers, diastereomers, racemates, zwitterions, cations, or mixtures thereof that occur.

The term “amino acid” is understood as the stereoisomeric forms, e.g. D- and L-forms, of the following compounds: asparagine, arginine, aspartic acid, glutamine, glutamic acid, β-alanine, γ-aminobutyrate, N_{ε}-acetyllysine, N_{δ}-acetylornithine, N_{γ}-acetyldiaminobutyrate, N_{α}-acetyldiaminobutyrate, histidine, isoleucine, leucine, methionine, phenylalanine, serine, threonine and tyrosine.

L-amino acids are preferred. Amino-acid radicals are derived from the corresponding amino acids. The following amino-acid radicals are preferred: Gly, Ala, Ser, Thr, Val, β-Ala, γ-aminobutyrate, Asp, Glu, Asn, Aln, N_{ε}-acetyllysine, N_{δ}-acetylornithine, N_{γ}-acetyldiaminobutyrate, N_{α}-acetyldiaminobutyrate.

The amino acids have been abbreviated in accordance with generally usual notation. The di- or tripeptide radicals are acid amides in terms of their chemical nature, and decompose into two or three amino acids upon hydrolysis. The amino acids in the di- or tripeptide radical are joined to one another by amide bonds.

With regard to the manufacture of di- and tripeptide radicals, reference is expressly made to EP 0 671 161 A1 of the Marbert company. Examples of di- and tripeptide radicals are also evident from the disclosure of EP 0 671 161 A1.

Examples of C₁ to C₄ alkyl groups in the compounds of formula (IV) are methyl, ethyl, n-propyl, isopropyl, n-butyl, isobutyl and tert.-butyl. Preferred alkyl groups are methyl and ethyl; methyl is a particularly preferred alkyl group. Preferred C₂ to C₄ hydroxyalkyl groups are the 2-hydroxyethyl, 3-hydroxypropyl or 4-hydroxybutyl groups; 2-hydroxyethyl is a particularly preferred hydroxyalkyl group.

These care-providing substances are used preferably in quantities from 0.001 to 2, in particular from 0.01 to 0.5 wt %, based in each case on the entire application preparation.

Mono- or oligosaccharides can also be used as a care-providing substance in the products according to the present invention.

Both monosaccharides and oligosaccharides, for example raw sugar, milk sugar, and raffinose, can be used. The use of monosaccharides is preferred according to the present invention. Among the monosaccharides, those compounds containing 5 or 6 carbon atoms are in turn preferred.

Suitable pentoses and hexoses are, for example, ribose, arabinose, xylose, lyxose, allose, altrose, glucose, mannose, gulose, idose, galactose, talose, fucose and fructose. Arabinose, glucose, galactose and fructose are carbohydrates that are preferably used; it is very particularly preferred to use glucose, which is suitable both in the D-(+) or L-(−) configuration or as a racemate.

Derivatives of these pentoses and hexoses, such as the corresponding -onic and -uronic acids (sugar acids), sugar alcohols, and glycosides, can also be used according to the present invention. Preferred sugar acids are gluconic acid, glucuronic acid, saccharic acid, mannosaccharic acid, and mucic acid. Preferred sugar alcohols are sorbitol, mannitol, and dulcitol. Preferred glycosides are the methylglucosides.

Because the mono- or oligosaccharides that are used are usually obtained from natural raw materials such as starch, in general they exhibit the configurations corresponding to those raw materials (e.g. D-glucose, D-fructose and D-galactose).

The mono- or oligosaccharides are contained in the products according to the present invention preferably in a quantity from 0.1 to 8 wt %, particularly preferably from 1 to 5 wt %, based on the entire application preparation.

Suitable care-providing substances are, furthermore, lipids.

Lipids suitable according to the present invention are phospholipids, for example soy lecithin, egg lecithin, and kephalins, as well as the substances known by the INCI names Linoleamidopropyl PG-Dimonium Chloride Phosphate, Cocamidopropyl PG-Dimonium Chloride Phosphate, and Stearamidopropyl PG-Dimonium Chloride Phosphate. These are marketed, for example, by the Mona company under the commercial designations Phospholipid EFA®, Phospholipid PTC®, and Phospholipid SV®.

The lipids are used preferably in quantities from 0.01 to 10 wt %, in particular 0.1 to 5 wt %, based on the entire application preparation.

Oily substances are also suitable as a care-providing substance.

Among the natural and synthetic cosmetic oily substances may be listed, for example;
Vegetable oils. Examples of such oils are sunflower oil, olive oil, soybean oil, rapeseed oil, almond oil, jojoba oil, orange oil, wheat germ oil, peach-kernel oil, and the liquid components of coconut oil. Also suitable, however, are other triglyceride oils such as the liquid components of beef tallow, as well as synthetic triglyceride oils.
Liquid paraffin oils, isoparaffin oils, and synthetic hydrocarbons, as well as di-n-alkyl ethers having a total of between 12 and 36 carbon atoms, in particular 12 to 24 carbon atoms, such as, for example, di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether, and n-hexyl-n-undecyl ether, as well as ditert.-butyl ether, diisopentyl ether, di-3-ethyldecyl ether, tert.-butyl-n-octyl ether, isopentyl-n-octyl ether, and 2-methylpentyl-n-octyl ether. The compounds 1,3-di-(2-ethylhexyl)cyclohexane (Cetiol® S) and di-n-octyl ether (Cetiol® OE), available as commercial products, may be preferred.
Ester oils. “Ester oils” are to be understood as the esters of C₆ to C₃₀ fatty acids with C₂ to C₃₀ fatty alcohols. The monoesters of fatty acids with alcohols having 2 to 24 carbon atoms are preferred. Examples of fatty acid components used in the esters are hexanoic acid, octanoic acid, 2-ethylhexanoic acid, decanoic acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeostearic acid, arachidic acid, gadoleic acid, behenic acid, and erucic acid, as well as industrial mixtures thereof that occur, for example, upon high-pressure cleavage of natural fats and oils, upon oxidation of aldehydes from Roelen oxosynthesis, or upon dimerization of unsaturated fatty acids. Examples of the fatty alcohol components in the ester oils are isopropyl alcohol, hexanol, octanol, 2-ethylhexyl alcohol, decanol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol, and brassidyl alcohol, as well as industrial mixtures thereof that occur, for example, upon high-pressure hydrogenation of industrial methyl esters based on fats and oils or aldehydes from Roelen oxosynthesis, and as a monomer fraction upon dimerization of unsaturated fatty alcohols. Particularly preferred according to the present invention are isopropyl myristate (Rilanit® IPM), isononanoic acid C₁₆₋₁₈ alkyl ester (Cetiol® SN), 2-ethylhexyl palmitate (Cegesoft® 24), stearic acid 2-ethylhexyl ester (Cetiol® 868), cetyl oleate, glycerol tricaprylate, coconut fatty alcohol caprinate/caprylate (Cetiol® LC), n-butyl stearate, oleyl erucate (Cetiol® J 600), isopropyl palmitate (Rilanit® IPP), Oleyl Oleate (Cetiol®), lauric acid hexyl ester (Cetiol® A), di-n-butyl adipate (Cetiol® B), myristyl myristate (Cetiol® MM), Cetearyl isononanoate (Cetiol® SN), oleic acid decyl ester (Cetiol® V).
Dicarboxylic acid esters such as di-n-butyl adipate, di(2-ethylhexyl)adipate, di(2-ethylhexyl)succinate, and diisotridecyl acelaate, as well as diol esters such as ethylene glycol dioleate, ethylene glycol diisotridecanoate, propylene glycol di(2-ethyl hexanoate), propylene glycol diisostearate, propylene glycol dipelargonate, butanediol diisostearate, neopentyl glycol dicaprylate.
Symmetrical, asymmetrical, or cyclic esters of carbonic acid with fatty alcohols, described for example in German Application DE 197 56 454, glycerol carbonate, or dicaprylyl carbonate (Cetiol® CC).
Fatty acid triesters of saturated and/or unsaturated linear and/or branched fatty acids with glycerol.
Fatty acid partial glycerides, to be understood as monoglycerides, diglycerides, and industrial mixtures thereof. When industrial products are used, small quantities of triglycerides can still be present for manufacturing-related reasons. The partial glycerides preferably conform to formula (D4-I):

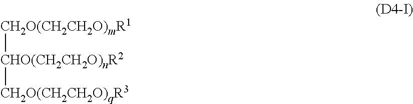

in which R¹, R² and R³, mutually independently, denote hydrogen or a linear or branched, saturated and/or unsaturated acyl radical having 6 to 22, preferably 12 to 18, carbon atoms, with the stipulation that at least one of these groups denotes an acyl radical and at least one of these groups denotes hydrogen. The sum (m+n+q) denotes 0 or numbers from 1 to 100, preferably 0 or 5 to 25. R¹ preferably denotes an acyl radical and R² and R³ denote hydrogen, and the sum (m+n+q) is 0. Typical examples are mono- and/or diglycerides based on hexanoic acid, octanoic acid, 2-ethylhexanoic acid, decanoic acid, lauric acid, isotridecanoic acid, myristic acid, palmitic acid, palmoleic acid, stearic acid, isostearic acid, oleic acid, elaidic acid, petroselinic acid, linoleic acid, linolenic acid, elaeostearic acid, arachidic acid, gadoleic acid, behenic acid and erucic acid, as well as industrial mixtures thereof. Oleic acid monoglycerides are used by preference.

The quantity of the natural and synthetic cosmetic oily substances used in the products according to the present invention is usually 0.1 to 30 wt % based on the entire application preparation, preferably 0.1 to 20 wt %, and in particular 0.1 to 15 wt %.

The product can furthermore contain an enzyme as a care-providing substance. Enzymes particularly preferred according to the present invention are selected from a group made up of proteases, lipases, transglutaminases, oxidases and peroxidases.

Pearl extracts are also suitable as a care-providing substance.

Mussel pearls are made up substantially of inorganic and organic calcium salts, trace elements, and proteins. Pearls can easily be obtained from cultivated mussels. Mussel cultivation can be accomplished in both fresh water and seawater; this can have an effect on the constituents of the pearls. A pearl extract that derives from mussels cultivated in seawater or salt water is preferred according to the present invention. The pearls are made up largely of aragonite (calcium carbonate), conchiolin, and an albuminoid; the latter constituents are proteins. Also contained in pearls are magnesium and sodium salts, inorganic silicon compounds, and phosphates.

The pearls are powdered for production of the pearl extract. The powdered pearls are then extracted with the usual methods. Water, alcohols, and mixtures thereof can be used as extraction agents for production of the pearl extracts. “Water” is to be understood in this context as both demineralized water and seawater. Among the alcohols, lower alcohols such as ethanol and isopropanol, but in particular polyvalent alcohols such as glycerol, diglycerol, triglycerol, polyglycerol, ethylene glycol, propylene glycol, and butylene glycol are preferred, both as a sole extraction agent and also mixed with demineralized water or seawater. Pearl extracts based on water/glycerol mixtures have proven to be particularly suitable. Depending on the extraction conditions, the pearl proteins (conchiolin and albuminoid) can be present to a very large extent in the natural state, or already partly or very largely as protein hydrolysates. A pearl extract in which conchiolin and albuminoid are already present in partly hydrolyzed fashion is preferred. The essential amino acids of these proteins are glutamic acid, serine, alanine, glycine, aspartic acid, and phenylalanine. In a further particularly preferred embodiment, it may be advantageous if the pearl extract is additionally enriched with at least one or more of these amino acids. In the most preferred embodiment, the pearl extract is enriched with glutamic acid, serine, and leucine. In addition, depending on the extraction conditions, in particular as a function of the extraction agent selected, a greater or lesser proportion of minerals and trace elements may still be present in the extract. A preferred extract contains organic and/or inorganic calcium salts as well as magnesium and sodium salts, inorganic silicon compounds, and/or phosphates. A very particularly preferred pearl extract contains at least 75%, preferably 85%, particularly preferably 90%, and very particularly preferably 95% of all the constituents of the naturally occurring pearls. Examples of pearl extracts usable according to the present invention are the commercial products Pearl Protein Extract BG® or Crodarom® Pearl.

The pearl extracts described above are contained preferably in a quantity from at least 0.01 to 20 wt %. The quantities of the extract used are preferably from 0.01 to 10 wt %, very particularly preferably 0.01 to 5 wt %, based on the entire product.

Although each of the aforesaid care-providing substances already yields a satisfactory result of itself, all embodiments in which the product contains multiple care-providing substances, including from different groups, are also encompassed within the scope of the present invention.

The products according to the present invention can moreover contain at least one UV filter.

The addition of a UV filter allows both the products themselves, and the treated fibers, to be protected from damaging influences of UV radiation. The UV filters suitable according to the present invention are not subject to any general restrictions in terms of their structure and their physical properties. Instead, all UV filters usable in the cosmetics sector, whose absorption maximum lies in the UVA (315 to 400 nm) UVB (280 to 315 nm), or UVC (<280 nm) regions, are suitable. UV filters having an absorption maximum in the UVB region, in particular in the region from approximately 280 to approximately 300 nm, are particularly preferred.

The UV filters preferred according to the present invention can be selected, for example, from substituted benzophenones, p-aminobenzoic acid esters, diphenylacrylic acid esters, cinnamic acid esters, salicylic acid esters, benzimidazoles, and o-aminobenzoic acid esters.

Examples of UV filters usable according to the present invention are 4-aminobenzoic acid, N,N,N-trimethyl-4(2-oxoborn-3-ylidenemethyl)aniline methylsulfate, 3,3,5-trimethylcyclohexyl salicylate (Homosalate), 2-hydroxy-4-methoxybenzophenone (Benzophenone-3; Uvinul® M 40, Uvasorb® MET, Neo Heliopan® BB, Eusolex® 4360), 2-phenylbenzimidazole-5-sulfonic acid and its potassium, sodium, and triethanolamine salts (phenylbenzimidazolesulfonic acid; Parsol® HS; Neo Heliopan® Hydro), 3,3′-(1,4-phenylenedimethylene)-bis(7,7-dimethyl-2-oxobicyclo-[2.2.1]hept-1-ylmethanesulfonic acid) and its salts, 1-(4-tert.-butylphenyl)-3-(4-methoxyphenyl)propane-1,3-dione (butylmethoxydibenzoylmethane; Parsol® 1789, Eusolex® 9020), α-(2-oxoborn-3-ylidene)toluene-4-sulfonic acid and its salts, ethoxylated 4-aminobenzoic acid ethyl ester (PEG-25 PABA; Uvinul® P 25), 4-dimethylaminobenzoic acid 2-ethylhexyl ester (Octyl Dimethyl PABA; Uvasorb® DMO, Escalol® 507, Eusolex® 6007), salicylic acid 2-ethylhexyl ester (Octyl Salicylate; Escalol® 587, Neo Heliopan® OS, Uvinul® 018), 4-methoxycinnamic acid isopentyl ester (isoamyl p-Methoxycinnamate; Neo Heliopan® E 1000), 4-methoxycinnamic acid 2-ethylhexyl ester (Octyl Methoxycinnamate; Parsol® MCX, Escalol® 557, Neo Heliopan® AV), 2-hydroxy-4-methoxybenzophenone-5-sulfonic acid and its sodium salt (Benzophenone-4; Uvinul® MS 40; Uvasorb® S 5), 3-(4′-methylbenzylidene) D,L-camphor (4-Methylbenzylidene Camphor; Parsol® 5000, Eusolex® 6300), 3-benzylidene camphor (3-Benzylidene Camphor), 4-isopropylbenzyl salicylate, 2,4,6-trianilino-(p-carbo-2′-ethylhexyl-1′-oxi)-1,3,5-triazine, 3-imidazol-4-ylacrylic acid and its ethyl esters, polymers of N-{(2 and 4)-[2-oxoborn-3-ylidenemethyl]benzyl}acrylamide, 2,4-dihydroxybenzophenone (Benzophenone-1; Uvasorb® 20 H, Uvinul® 400), 1,1′-diphenylacrylonitrilic acid 2-ethylhexyl ester (Octocrylene, Eusolex® OCR, Neo Heliopan® Type 303, Uvinul® N 539 SG), o-aminobenzoic acid menthyl ester (Menthyl Anthranilate; Neo Heliopan® MA), 2,2′,4,4′-tetrahydroxybenzophenone (Benzophenone-2; Uvinul® D-50), 2,2′-dihydroxy-4,4′-dimethoxybenzophenone (Benzophenone-6), 2,2′-dihydroxy-4,4′-dimethoxybenzophenone-5-sodiumsulfonate, and 2-cyano-3,3-diphenylacrylic acid 2′-ethylhexyl ester. 4-Aminobenzoic acid, N,N,N-trimethyl-4-(2-oxoborn-3-ylidenemethyl)aniline methylsulfate, 3,3,5-trimethylcyclohexyl salicylate, 2-hydroxy-4-methoxybenzophenone, 2-phenylbenzimidazole-5-sulfonic acid and its potassium, sodium, and triethanolamine salts, 3,3′-(1,4-phenylenedimethylene)-bis(7,7-dimethyl-2-oxo-bicyclo-[2.2.1]hept-1-ylmethanesulfonic acid) and its salts, 1-(4-tert.-butylphenyl)-3-(4-methoxyphenyl)propane-1,3-dione, α-(2-oxoborn-3-ylidene)toluene-4-sulfonic acid and its salts, ethoxylated 4-aminobenzoic acid ethyl ester, 4-dimethylaminobenzoic acid 2-ethylhexyl ester, salicylic acid 2-ethylhexyl ester, 4-methoxycinnamic acid isopentyl ester, 4-methoxycinnamic acid 2-ethylhexyl ester, 2-hydroxy-4-methoxybenzophenone-5-sulfonic acid and its sodium salt, 3-(4′-methylbenzylidene) D,L-camphor, 3-benzylidene camphor, 4-isopropylbenzyl salicylate, 2,4,6-trianilino-(p-carbo-2′-ethylhexyl-1′-oxi)-1,3,5-triazine, 3imidazol-4-ylacrylic acid and its ethyl esters, and polymers of N-{(2 and 4)-[2-oxoborn-3-ylidenemethyl]benzyl}acrylamide are preferred. Very particular preferred according to the present invention are 2-hydroxy-4-methoxybenzophenone, methoxybenzophenone, 2-phenylbenzimidazole-5-sulfonic acid and its potassium, sodium, and triethanolamine salts, 1-(4-tert.butylphenyl)-3-(4-methoxyphenyl)propane-1,3-dione, 4-methoxycinnamic acid 2-ethylhexyl ester, and 3-(4′-methylbenzylidene) D,L-camphor.

Those UV filters whose molar extinction coefficient at the absorption maximum is above 15,000, in particular above 20,000, are preferred.

With structurally similar UV filters, in many cases the water-insoluble compound exhibits the greater effectiveness as compared with water-soluble compounds of this kind that differ from it by having one or more additionally ionic groups. In the context of the invention, those UV filters of which no more than 1 wt %, in particular no more than 0.1 wt %, dissolves in water at 20° C., are understood as water-insoluble. These compounds should furthermore be soluble at a proportion of at least 0.1 wt %, in particular at least 1 wt %, in common cosmetic oil components at room temperature. The use of water-insoluble UV filters can therefore be preferred according to the present invention.

According to a further embodiment of the present invention, those UV filters that comprise a cationic group, in particular a quaternary ammonium group, are preferred.

These UV filters exhibit the general structure U-Q.

The structural part U denotes a group that absorbs UV radiation. This group can be derived in principle from the aforementioned known UV filters usable in the cosmetics sector, in which one group, generally a hydrogen atom, of the UV filter is replaced by a cationic group Q, in particular by a quaternary amino function.

Compounds from which the structural part U can be derived are, for example
substituted benzophenones;
p-aminobenzoic acid esters;
diphenylacrylic acid esters;
cinnamic acid esters;
salicylic acid esters;
benzimidazoles; and
o-aminobenzoic acid esters.

The structural parts U that are derived from cinnamic acid amide or from N,N-dimethylaminobenzoic acid amide are preferred according to the present invention.

The structural parts U can in principle be selected so that the absorption maximum of the UV filters can lie both in the UVA (315 to 400 nm) region and in the UVB (280 to 315 nm) region, or in the UVC (<280 nm) region. UV filters having an absorption maximum in the UVB region, in particular in the region from approximately 280 to approximately 300 nm, are particularly preferred.

The structural part U is furthermore preferably selected, including as a function of the structural part Q, in such a way that the molar extinction coefficient of the UV filter at the absorption maximum is above 15,000, in particular above 20,000.

The structural part Q preferably contains a quaternary ammonium group as a cationic group. This quaternary ammonium group can in principle be connected directly to the structural part U, so that the structural part U represents one of the four substituents of the positively charged nitrogen atom. Preferably, however, one of the four substituents on the positively charged nitrogen atom is a group, in particular an alkylene group, having 2 to 6 carbon atoms, that functions as a connection between the structural part U and the positively charged nitrogen atom.

Advantageously, the group Q has the general structure —(CH₂)_{X}—N⁺R¹R²R³ X⁻, in which x denotes an integer from 1 to 4, R¹ and R², mutually independently, denote C₁₋₄ alkyl groups, R³ denotes a C₁₋₂₂ alkyl group or a benzyl group, and X⁻ denotes a physiologically acceptable anion. In the context of this general structure, x preferably denotes the number 3, R¹ and R² each denote a methyl group, and R³ denotes either a methyl group or a saturated or unsaturated, linear or branched hydrocarbon chain having 8 to 22, in particular 10 to 18, carbon atoms.

Physiologically acceptable anions are, for example, inorganic anions such as halides, in particular chloride, bromide and fluoride, sulfate ions, and phosphate ions, as well as organic anions such as lactate, citrate, acetate, tartrate, methosulfate, and tosylate.

Two preferred UV filters having cationic groups are the compounds cinnamic acid amidopropyltrimethylammonium chloride (Incroquat® UV-283) and dodecyldimethylaminobenzamidopropyldimethylammonium tosylate (Escalol® HP 610), available as commercial products.

The teaching of the present invention of course also encompasses the use of a combination of several UV filters. In this case, the combination of at least one water-insoluble UV filter with at least one UV filter having a cationic group is preferred.

The UV filters are usually contained in quantities from 0.01 to 5 wt % based on the entire application preparation. Quantities from 0.1 to 2.5 wt % are preferred.

Further active substances, adjuvants, and additives are, for example:
thickening agents such as agar-agar, guar gum, alginates, xanthan gum, gum arabic, karaya gum, locust bean flour, linseed gums, dextrans, cellulose derivatives, e.g. methyl cellulose, hydroxyalkyl cellulose, and carboxymethyl cellulose, starch fractions and derivatives such as amylose, amylopectin, and dextrins, clays such as e.g. bentonite, or entirely synthetic hydrocolloids such as e.g. poly(vinyl alcohol),
structuring agents such as maleic acid and lactic acid,
perfume oils, dimethyl isosorbide, and cyclodextrins,
solvents and solubilizers such as ethanol, isopropanol, ethylene glycol, propylene glycol, glycerol, and diethylene glycol,
quaternized amines such as methyl-1-alkylamidoethyl-2-alkylimidazolinium methosulfate,
dyes for coloring the agent,
anti-dandruff ingredients such as piroctone olamine, zinc omadine, and climbazol,
cholesterol,
consistency agents such as sugar esters, polyol esters, or polyol alkyl ethers,
fats and waxes such as spermaceti, beeswax, montan wax, and paraffins,
fatty acid alkanolamides,
complexing agents such as EDTA, NTA, β-alaninediacetic acid, and phosphonic acids,
swelling and penetrating substances such as glycerol, propylene glycol monoethyl ether, carbonates, hydrogencarbonates, guanidine, ureas, as well as primary, secondary, and tertiary phosphates,
opacifiers such as latex, styrene/PVP and styrene/acrylamide copolymers
luster agents such as ethylene glycol mono- and distearate, as well as PEG-3 distearate,
preservatives,
stabilizing agents for hydrogen peroxide and other oxidizing agents,
antioxidants.

If the products according to the present invention involve an aerosol-foam product, the latter mandatorily contains a propellant.

Propellants suitable according to the present invention are, for example, N₂O, dimethyl ether, CO₂, air, and alkanes having 3 to 5 carbon atoms, such as propane, n-butane, isobutane, n-pentane, and isopentane, and mixtures thereof. Dimethyl ether, propane, n-butane, isobutanes, and mixtures thereof are preferred.

According to a preferred embodiment, the aforesaid alkanes, mixtures of the aforesaid alkanes, or mixtures of the aforesaid alkanes with dimethyl ether are used as the only propellant. The invention also expressly encompasses, however, the concurrent use of propellants of the chlorofluorocarbon type, but in particular the fluorocarbons.

For a given spray apparatus, the sizes of the foam bubbles and their respective size distribution, and thus also the foam stability, can be influenced by way of the quantitative ratio between the propellant and the other constituents of the preparations.

When conventional spray apparatuses are used, the aerosol-foam products according to the present invention contain the propellant preferably in quantities from 1 to 50 wt %, based on the entire product. Quantities from 5 to 30 wt %, in particular from 5 to 20 wt %, are particularly preferred.

The products according to the present invention can be both hair treatment agents that are rinsed out of the hair after application and a certain contact time (i.e. so-called rinse-off products), and products that remain in the hair after application (i.e. so-called leave-on products). Because, however, depending on the film-forming and/or setting polymer, the desired setting effect in particular declines considerably or is even completely eliminated by rinsing out the product, the products according to the present invention are by preference leave-on products.

The Examples that follow are intended to explain the subject matter of the present invention without limiting it in any way.

### EXAMPLES

The following formulations were manufactured. Unless otherwise noted, the quantitative indications are understood as percentages by weight; indications of pH refer to 25° C.

### 1 Tinting Foams

Firstly, the foamable compositions E1 to E11 were manufactured by mixing the raw materials recited in Tables 1 and 2. The compositions exhibited a pH of between 5.5 and 6.0.

TABLE 1

| Raw materials | E1 | E2 | E3 | E4 | E5 |
|---|---|---|---|---|---|
| Natrosol 250 HR¹ | 0.2 | 0.2 | 0.2 | 0.2 | 0.2 |
| Luviskol VA 64 W² | 5.5 | 5.5 | 5.5 | 5.5 | 5.5 |
| Luviquat FC 370³ | 2.8 | 2.8 | 2.8 | 2.8 | 2.8 |
| Luviquat Mono CP⁴ | 1.1 | 1.1 | 1.1 | 1.1 | 1.1 |
| Sodium benzoate | 0.3 | 0.3 | 0.3 | 0.3 | 0.3 |
| D-panthenol 75% | 0.2 | 0.2 | 0.2 | 0.2 | 0.2 |
| PEG-40 Hydrogenated | 0.2 | 0.2 | 0.2 | 0.2 | 0.2 |
| Castor Oil⁵ | | | | | |
| Perfume | 0.2 | 0.2 | 0.2 | 0.2 | 0.2 |
| Citric acid monohydrate | 0.01 | 0.01 | 0.01 | 0.01 | 0.01 |
| Acid Violet 43 | 0.05 | 0.08 | — | 0.02 | 0.20 |
| Acid Black 1 | — | — | 0.04 | — | — |
| Basic Brown 16 | — | — | — | 0.01 | 0.02 |
| Basic Brown 17 | 0.1 | 0.2 | 0.2 | 0.01 | 0.08 |
| Water, deionized | to make | to make | to make | to make | to make |
| | 100 | 100 | 100 | 100 | 100 |

¹Hydroxyethylcellulose (INCI name: Hydroxyethylcellulose) (Hercules)
²Vinylpyrrolidone/vinyl acetate copolymer (60:40) (approx. 48 to 52% solids in water; INCI name: VP/VA Copolymer) (BASF)
³3-Methyl-1-vinylimidazolium chloride/vinylpyrrolidone copolymer (30:70) (38 to 42% solids in water; INCI name: Polyquaternium-16) (BASF)
⁴Hexadecyl(2-hydroxyethyl)dimethylammonium dihydrogenphosphate (27 to 33% solids in water; INCI name: Hydroxyethyl Cetyldimonium Phosphate) (BASF)
⁵Polyethylene glycol derivative of hydrogenated castor oil having an average of 40 mol ethylene oxide (INCI name: PEG-40 Hydrogenated Castor Oil) (BASF)

TABLE 2

| Raw materials | E6 | E7 | E8 | E9 | E10 | E11 |
|---|---|---|---|---|---|---|
| Natrosol 250 HR¹ | 0.2 | 0.2 | 0.2 | 0.2 | 0.2 | 0.2 |
| Luviskol VA | 5.5 | 5.5 | 5.5 | 5.5 | 5.5 | 5.5 |
| 64 W² | | | | | | |
| Luviquat FC 370³ | 2.8 | 2.8 | 2.8 | 2.8 | 2.8 | 2.8 |
| Luviquat Mono | 1.1 | 1.1 | 1.1 | 1.1 | 1.1 | 1.1 |
| CP⁴ | | | | | | |
| Sodium benzoate | 0.3 | 0.3 | 0.3 | 0.3 | 0.3 | 0.3 |
| D-panthenol 75% | 0.2 | 0.2 | 0.2 | 0.2 | 0.2 | 0.2 |
| PEG-40 | 0.2 | 0.2 | 0.2 | 0.2 | 0.2 | 0.2 |
| Hydrogenated | | | | | | |
| Castor Oil⁵ | | | | | | |
| Perfume | 0.2 | 0.2 | 0.2 | 0.2 | 0.2 | 0.2 |
| Citric acid | 0.01 | 0.01 | 0.01 | 0.01 | 0.01 | 0.01 |
| monohydrate | | | | | | |
| Acid Violet 43 | 0.1 | 0.15 | 0.2 | — | 0.1 | 0.1 |
| Acid Blue 9 | — | — | — | 0.25 | — | — |
| Acid Blue 62 | — | — | — | — | — | 0.01 |
| Acid Black 1 | — | — | — | — | — | 0.01 |
| Basic Brown 16 | — | 0.02 | — | — | — | — |
| Basic Brown 17 | — | 0.06 | — | — | — | — |
| Basic Yellow 57 | 0.05 | — | — | — | — | — |
| Basic Yellow 87 | — | — | — | — | 0.05 | 0.05 |
| Basic Violet 2 | — | — | 0.04 | 0.04 | — | — |
| Red 33 | — | 0.0004 | — | — | — | |
| Water, deionized | to | to make | to make | to make | to make | to make |
| | make | 100 | 100 | 100 | 100 | 100 |
| | 100 | | | | | |

The foamable compositions E1 to E11 were each introduced in usual fashion into pressure-resistant aerosol containers; once the container had been sealed and provided with a valve, a propellant mixture of n-propane, n-butane, and isobutane (48/49/3) was added at a weight ratio of 9:1.

Investigations of shelf stability were performed both at 25° C. and at 45° C. In both cases, no problems at all with stability of the formulations were observed in the context of storage over a period of 3 months. There were also no changes, within the period of time indicated, in the color result when the products were applied.

### 2 Application

The desired quantity of tinting foam was removed from each of the containers and incorporated uniformly into freshly washed, towel-dried hair. The hair was then dried with a hot-air dryer, in which context the desired hairstyle was simultaneously formed. A temporary change in shape and color was thereby achieved. The hue was determined in each case under a daylight lamp (HE240A color tester) and scored (Taschenlexikon der Farben [Pocket color dictionary], A. Kornerup and J. H. Wanscher, 3rd unmodified edition 1981, Muster-Schmidt Verlag; Zürich, Göttingen), and is reproduced in Table 3.

TABLE 3

| Composition | Hue |
|---|---|
| E1 | Medium blonde |
| E2 | Dark blonde |
| E3 | Natural dark blonde |
| E4 | Beige |
| E5 | Anthracite |
| E6 | Silver |
| E7 | Gray |
| E8 | Violet |
| E9 | Dark violet |
| E10 | Silver |
| E11 | Dark silver |

In all cases, colors notable for a neutral color tendency were obtained.

1. An aerosol-foam product or pump-foam product for the treatment of keratinic fibers, encompassing a foam-type or foamable composition comprising, in a cosmetically acceptable carrier,
a) a polymer selected from the group consisting of film-forming polymers and setting polymers, and
b) an anionic direct dye.

2. The product of claim 1, wherein the foam-type or foamable composition comprises the polymer in a quantity from 0.5 to 15 wt %.

3. The product of claim 1, wherein the polymer comprises a nonionic polymer selected from the group consisting of film-forming polymers and setting polymers.

4. The product of claim 3, wherein the nonionic polymer is a homo- or copolymer of vinylpyrrolidone.

5. The product of claim 1, wherein the polymer comprises a cationic polymer selected from the group consisting of film-forming polymers and setting polymers.

6. The product of claim 5, wherein the cationic polymer is a quaternized homo- or polymer of dimethyldiallylammonium chloride or a copolymer of vinylpyrrolidone with at least one further monomer, which optionally after quaternization comprises at least one cationic group.

7. The product of claim 1, wherein the anionic direct dye is selected from the group consisting of blue- or violet-coloring dyes.

8. The product of claim 7, wherein the anionic direct dye is selected from the group consisting of (2-sulfophenyl)di[4-(ethyl((4-sulfophenyl)methyl)amino)phenyl]carbenium disodium salt betaine (C.I. 42,090; Acid Blue 9; FD&C Blue No. 1), 1-amino-4-(cyclohexylamino)-9,10-anthraquinone-2-sulfonic acid sodium salt (C.I. 62,045; Acid Blue 62), 1-hydroxy-4-[(4-methyl-2-sulfophenyl)amino]-9,10-anthraquinone sodium salt (C.I. 60,730; D&C Violet No, 2; Acid Violet 43), and 5-amino-4-hydroxy-6-[(4-nitrophenyl)azo]-3-(phenylazo)-2,7-naphthalenedisulfonic acid disodium salt (C.I. 20,470; Acid Black 1).

9. The product of claim 8, wherein the anionic direct dye is 1-hydroxy-4-[(4-methyl-2-sulfophenyl)amino]-9,10-anthraquinone sodium salt (C.I. 60,730; D&C Violet No. 2; Acid Violet 43).

10. The product of claim 1, wherein the foam-type or foamable composition further comprises a cationic direct dye.

11. The product of claim 1, wherein the foam-type or foamable composition further comprises a quaternary ammonium compound.

12. The product of claim 11, wherein the quaternary ammonium compound is a compound of formula (Q-I) 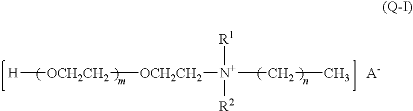
in which
R¹ and R², mutually independently in each case, denote an optionally hydroxy-substituted C₁ to C₄ alkyl group,
m denotes an integer from 0 to 20,
n denotes an integer from 0 to 20, and
A⁻ denotes a monovalent anion.

13. The product of claim 12, wherein in the compound of formula (Q-I), R¹ and R² denote methyl, m denotes 0, n denotes an integer from 9 to 17, and A⁻ denotes a monovalent anion.

14. The product of claim 1, wherein its pH is less than 7.

15. The product of claim 14, wherein its pH is from 5.0 to 6.5.

16. The product of claim 1, wherein the foam-type or foamable composition comprises the polymer in a quantity from 1.0 to 10 wt %.
